(19) United States
(12) Patent Application Publication
(10) Pub. No.: US 2014/0349378 A1
(43) Pub. Date: Nov. 27, 2014

(54) Biomass Fertilization Vessels

(71) Applicant: Coastal Waters Biotechnology Group LLC, San Jose, CA (US)

(72) Inventor: Dennis D. Yancey, San Jose, CA (US)

(73) Assignee: Coastal Waters Biotechnology Group LLC, San Jose, CA (US)

(21) Appl. No.: 14/294,001

(22) Filed: Jun. 2, 2014

Related U.S. Application Data
(63) Continuation of application No. PCT/US2012/067448, filed on Nov. 30, 2012.
(60) Provisional application No. 61/566,519, filed on Dec. 2, 2011.
(60) Provisional application No. 61/700,884, filed on Sep. 14, 2012.

(51) Int. Cl. C12N 1/12

(52) U.S. Cl. CPC C12N 1/12
USPC 435/257.1; 435/283.1

(57) ABSTRACT

A system for collecting and/or harvesting biomass comprises a first module adapted to accept a fluid stream. The first module includes a gate that is adapted to regulate the flow of the fluid stream. The system includes a second module downstream of the first module. The second module accepts a fluid from the first module and directs at least a portion of the fluid to the first module. A third module downstream of the second module includes one or more surfaces for retaining one or more microorganisms upon the flow of at least a portion of the fluid stream through the third module.

### CROSS-REFERENCE

This application is a continuation application of PCT/US2012/067448, filed Nov. 30, 2012, which claims the benefit of U.S. Provisional Application No. 61/566,519, filed Dec. 2, 2011, and U.S. Provisional Application No. 61/700,884, filed Sep. 14, 2012, each of which is entirely incorporated herein by reference.

### BACKGROUND

On a global scale, the continued use of fossil fuels is now widely accepted as unsustainable due to environmental factors. According to National Oceanic and Atmospheric Administration's Earth System Research Laboratory, the depletion of resources and accumulation of greenhouse gases in the environment have already exceeded the “dangerously high” threshold of 390 ppm CO₂ equivalents. On a national scale, the reduction of energy dependency on foreign oil is now viewed as essential to ensuring the long-term national security and economic stability of the United States. To achieve environmental and economic sustainability, fuel production processes must be both renewable and capable of sequestering atmospheric CO₂. Currently, nearly all renewable energy sources (e.g., hydroelectric, solar, wind, tidal, geothermal) target the electricity market, leaving an expansive opportunity to develop alternative methodologies to produce renewable energy sources in the liquid fuels market. In fact, liquid fuels make up a much larger share of the global energy demand (66%); thus, the potential impact of developing renewable fuel solutions, such as biofuel production, in this sector is substantial.

There are several widely used biofuel production sources: algae biomass, corn bioethanol, and soy-based biodiesel. Biofuel production via algae biomass has several competitive advantages over corn bioethanol and soy-based biodiesel.³ Unlike the corn and soy biofuels derivatives, algae can be the source of a wide range of feedstocks for transformation into biodiesel, green diesel, ethanol, methane, and other fuels. Algae cultivation can take place in non-productive lands such as deserts and oceans. It is a non-food resource, and therefore does not compete with agricultural production. Algae cultivars may also be implemented in conjunction with CO₂-producing plants for in-situ carbon sequestration, which would be highly advantageous in a carbon cap-and-trade or carbon credit economy. Finally, algae is widely regarded as one of the most efficient ways of generating biofuels, thanks to a 50-fold increase in theoretical energy yield compared to traditional crops.

Despite the enormous potential of algae, from a commercialization analysis, scenarios for producing substantial amounts of transportation fuels (e.g. diesel and ethanol) from microalgae require a strategic production process that minimizes costs while both maximizing biomass yields and optimizing the various processes involved in conversion to fuels. To date, production process research has focused on cultivating genetically modified algae strains that have been engineered to improve oil quality/quantity and generate proteins for the nutraceutical and the specialty green chemical production. Terrestrial algae cultivation in open raceway ponds is the current state of the art energy and cost efficient method for biomass production for biofuels, green chemicals, and nutraceuticals. Even with those efficiencies, profitability at mass production levels requires further reductions in energy consumption and costs for it to be viable at today's crude oil prices. As such, even large-scale biomass production costs with existing processes are still at least 10-times higher than equivalent processes for fossil sources. This cost difference is mainly due to the costs of makeup water lost from evaporation, nutrient input, susceptibility to invasive species, and climate variability, as well as variable costs reliant upon sector regulation, technologies developed and adopted, and national and international economic conditions impacting labor, energy, and capital costs. Furthermore, the cost of oil extraction from microalgae biomass remains high and is responsible for half of its total production cost.

### SUMMARY

This disclosure provides methods for cultivating microalgae in a body of water (e.g., the open ocean) and producing a steady and inexpensive feedstock stream. Methods are provided that use using ocean environments take advantage of the (A) free nutrients offered by the ocean, (B) free kinetic energy for mixing, (C) free organism cooling and hydration, (D) use of the ocean's vast underutilized surface area to overcome scaling limitations, (E) portability and ease of implementation in most ocean environments, and (F) a strategic intention to maintain partnerships with marine biology facilities to streamline processes for acquiring open water permits until its infrastructure and sector credibility has been established. Methods of the disclosure provide for generating renewable fuel feedstock that may be significantly cheaper than terrestrial cultivation sources, yet an energetically equivalent, fossil fuel substitute after catalytic hydroprocessing.

Methods provided herein may be more ecologically friendly than land based algal oil production. With methods and systems provided herein, it is possible to cultivate commercial scale algae biomass blooms in large containers, while minimizing waste streams.

An aspect of the disclosure provides a collector of biomass, comprising a vessel comprising (i) one or more surfaces for collecting a biomass from a fluid directed through the vessel, and (ii) one or more internal impellers in fluid communication with the one or more surfaces through a fluid flow path. The one or more internal impellers facilitate flow of the fluid through the fluid flow path. The collector further comprises an external impeller coupled to the one or more internal impellers. The external impeller is disposed external to the vessel and adapted to provide rotational energy to the internal impeller upon fluid flow through or adjacent to the external impeller. The vessel can include a housing having one or more modules. In an embodiment, the one or more surfaces are part of one or more plates. In another embodiment, the vessel comprises a magnet or electromagnet that is at least partially enclosed by an exterior wall of the vessel. In another embodiment, the vessel further comprises a self-orienting mechanism capable of orienting the direction of the vessel with respect to the direction of a current flow when the vessel is positioned in a current. In another embodiment, the fluid flow path is a circulatory fluid flow path. In another embodiment, the vessel comprises a first module and a second module, wherein the second module is downstream of the first module along the fluid flow path extending from the first module to the second module, and wherein the first module includes the one or more internal impellers. In another embodiment, the vessel further comprises a gate that at least partially isolates the fluid flow path. In another embodiment, the gate is a movable gate. In another embodiment, the vessel further comprises a membrane/sieve in the first module, wherein the membrane/sieve is included in the fluid flow path. In another embodiment, the vessel comprises a plurality of modules, and wherein the one or more surface and the one or more internal impellers are disposed in separate modules.

Another aspect provides a system for collecting and/or harvesting biomass, comprising a first module adapted to accept a fluid stream. The first module includes a gate that is adapted to regulate the flow of the fluid stream. The system further comprises a second module downstream of the first module. The second module accepts a fluid from the first module and directs at least a portion of the fluid to the first module. The system further comprises a third module downstream of the second module. The third module includes one or more surfaces for retaining one or more microorganisms upon the flow of at least a portion of the fluid stream through the third module. In an embodiment, the one or more surfaces are part of one or more plates. In another embodiment, the first module and second module are separable from one another. In another embodiment, the second module and third module are separable from one another. In another embodiment, the first module includes one or more impellers for facilitating fluid flow through the first and second modules. In another embodiment, the system further comprises an external impeller that is external to the first, second and third modules, wherein the external impeller is coupled to the internal impeller and imparts rotational motion to the one or more internal impellers upon fluid flow through or adjacent to the external impeller. In another embodiment, the system further comprises a fourth module between the first and second modules, wherein the fourth module extends a length of a fluid flow path from the first module to the second module. In another embodiment, the fourth module includes an optical window for permitting electromagnetic radiation for coming in contact with at least a portion of the fluid stream. In another embodiment, the system further comprises a nutrient concentrator upstream of the first module, wherein the nutrient concentrator is adapted to concentrate one or more nutrients in the fluid stream prior to the fluid stream entering the first module. In another embodiment, the nutrient concentrator includes a magnetic field source that is adapted to induce a magnetic force that concentrates the one or more nutrients.

Another aspect provides a method for collecting and/or harvesting biomass, comprising directing a fluid stream from a first module to a second module along a first fluid flow path leading from the first module to the second module. The fluid stream is directed through a movable gate of the first module. The movable gate is adapted to regulate fluid flow (i) along the first fluid flow path and (ii) along a second fluid flow path leading from the second module to the first module. Next, at least a portion of the fluid is directed from the second module to the first module along the second fluid flow path. Next, at least a portion of the fluid from the second module is directed to a third module. The third module includes one or more surfaces for retaining one or more microorganisms upon the flow of the at least the portion of the fluid through the third module. In an embodiment, the first fluid flow path is separate from the second fluid flow path. In another embodiment, the one or more surfaces are part of one or more plates. In another embodiment, the first module and second module are separable from one another. In another embodiment, the second module and third module are separable from one another. In another embodiment, the first module includes one or more internal impellers for facilitating fluid flow through the first and second modules. In another embodiment, the one or more internal impellers are coupled to an external impeller that is external to the first, second and third modules, wherein the external impeller imparts rotational motion to the one or more internal impellers upon fluid flow through or adjacent to the external impeller. In another embodiment, directing the fluid stream from the first module to the second module further comprises directing the fluid stream through a fourth module disposed between the first and second modules. In another embodiment, the fourth module includes an optical window for permitting electromagnetic radiation for coming in contact with at least a portion of the fluid stream.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF DRAWINGS

The novel features of the claimed invention are set forth with particularity in the appended claims. A better understanding of the features and advantages of the present invention will be obtained by reference to the following detailed description that sets forth illustrative embodiments, in which the principles of the invention are utilized, and the accompanying drawings or figures (also “FIG.” or “FIGs.” herein) of which:

FIG. 1 is a map identifying locations of iron fertilization experiments;

FIG. 2 is a plot that illustrates that nitrate concentration increases with depth;

FIG. 3A shows a vessel design incorporating baffles to circulate the microalgae through the vessel. FIG. 3B shows an offshore aquaculture implementation to generate renewable hydrocarbon fuels and proteins;

FIG. 4A is a schematic side view of a system (vessel) for collecting biomass; FIG. 4B is a schematic perspective view of the vessel of FIG. 4A;

FIGS. 5A-5C schematically illustrate a fertilization vessel;

FIGS. 6A-6D schematically illustrate a vessel with a forced conduction module; FIG. 6E is a schematic cross-section side view of impellers of the vessel of FIGS. 6A-6D;

FIG. 7 schematically illustrates the effect of the Lorenz force in a magnetic field on charged particles, in accordance with an embodiment of the invention;

FIG. 8 schematically illustrates a vessel that includes a bioharvester module and a magneto-concentrator module;

FIG. 9 schematically illustrates a magneto-concentrator module;

FIG. 10 provides another view of the magneto-concentrator module of FIG. 9;

FIG. 11 provides another view of a magneto-concentrator module of FIGS. 9 and 10;

FIG. 12 schematically illustrates a fertilizer recycler;

FIG. 13 provides another view of the fertilizer recycler of FIG. 12;

FIGS. 14A-14D show an example magneto hydro fertilizer concentrator;

FIG. 15 is a map showing the locations of oil rigs off of the United States Gulf coasts;

FIG. 16 shows an approach for scaling up a bioharvester, which may be any of the vessels of the disclosure;

FIG. 17 shows an aerial view of a typical open raceway pond (ORP) system;

FIG. 18 is a process flow diagram for cultivating biomass using vessels of the disclosure (CWBG) as compared to a typical ORP system;

FIG. 19 shows a table with energy savings using vessels of the disclosure (CWBG) as compared to ORP systems;

FIG. 20 shows a device for concentrating nitrate and phosphate anions along the length of an anode by applying a current orthogonal to the flow of water; and

FIG. 21A shows a growth profile of nannochloropsis oculata; FIG. 21B shows a light microscope image of the nannochloropsis oculata of FIG. 21A.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The terms “downstream” and “upstream,” as used herein, generally refer to the position of device or system components (e.g., modules) along a fluid flow path. For example, a first module downstream of a second module can be further along a fluid flow path than the second module, either in the same device or separate devices. The positions of the modules can be during instantaneous fluid flow. In some cases, during fluid flow in one general direction the first module is downstream of the second module, and when the general fluid direction is reversed, the first module can be upstream of the second module.

### Fertilization

Microalgae may require iron to assist in converting carbon dioxide into sugars using sunlight. Trace metals, such as iron, may be the key to inducing microalgae growth in ocean environments. The ocean's iron concentrations are generally well below the levels required to induce exponential growth in marine algae. As a result, marine microalgae are by and large in a stationary phase until storms or other natural events transport iron from land sources to coastal waters. Improving iron distribution in the ocean through human intervention would induce massive algal blooms and open the door to a sustainable and cost effective opportunity to produce green fuels, chemicals, and nutritional supplements in ocean environments that are normally barren. Past uncontrolled iron fertilization experiments have proven that artificially elevating iron concentrations in the open ocean is one of several keys to inducing marine microalgae growth that can be used as a feedstock for green products on an industrial scale

In some examples, microalgae growth may be induced by adding iron sulfate to 0.7 nM in a 225 km² area in the arctic polar front zone of the southern seas. The microalgae may cover approximate 2400 km² after 20 days of growth. Methods and systems of the disclosure may used induced microalgae as a renewable fuel feedstock.

### Low Nitrate and Phosphate Surface Waters

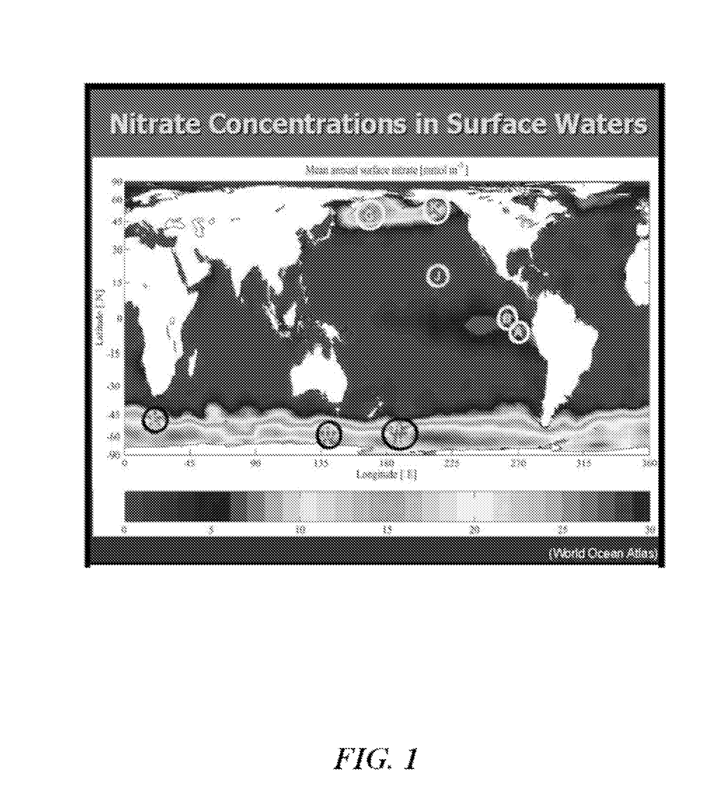

Most of the ocean's surface waters and other nutrients (nitrogen and phosphorous) are the primary limiters of algal productivity. FIG. 1 identifies where iron fertilization experiments are conducted. These sites are ideal for growing algae with iron fertilization because of the surface water's moderately high nitrate concentrations due to naturally occurring upwelling events. However, the locations identified in FIG. 1 are less than ideal as renewable fuel generation sites because of the significant distance from various economic markets or populated areas. For instance, ideal sites for the United States markets would be less than 200 km off the coast of metropolitan cities in calm sunny waters. Sites located near coastal metropolitan cities are ideal because they contain a dense concentration of end users of the renewable diesel, chemical, and nutraceutical products.

Systems of the disclosure may operate, in some cases, with the aid of open water nitrogen and phosphate sources that can be sequestered and concentrated to support high-density cultures. In systems containing ambient nitrate and phosphate concentrations, the volumetric production of algae can be low. Approaches for increasing surface water nitrate and phosphate concentration include, without limitation: (1) using terrigenous sources and/or (2) pumping nutrients from the deep ocean.

This disclosure provides systems and methods for retrieving (e.g., pumping) nutrients from the ocean at various depths, such as the deep ocean. This is based on the unexpected realization that the ocean's nitrate and phosphate concentration profiles not only vary along the surface, but also vary with depth, establishing in effect a 3-dimensional profile. The concentration of untapped nutrients in coastal waters at depths greater than 200 m is significant

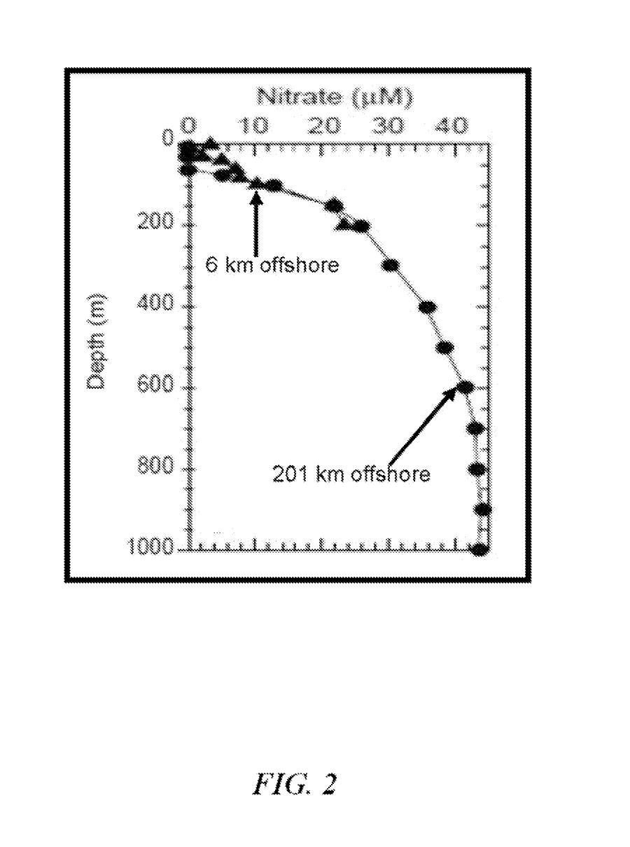

Although the nitrate surface concentrations in temperate and tropical zones are extremely low away from the coast, FIG. 2 illustrates that nitrate concentrations increase dramatically with depth. Based on FIG. 2, the nitrate concentrations between 150 m and 300 m are comparable to those on the surface in the Southern Seas. The concentrations of phosphates in the ocean are very highly correlated with nitrate concentrations, except at low oxygen values when nitrate may be lost as bacteria use nitrate as a terminal electron acceptor when oxidizing organic carbon. Based on these nitrate and phosphate depth concentration profiles, the macronutrient requirements needed to reproduce and exceed previous iron fertilization results are accessible by artificially “upwelling” phosphate and nitrates near the aquaculture site. Alternative nitrate and phosphate rich sources near coastal metropolitan cities are the rivers and tributaries that feed estuaries. These macronutrient sources can be precipitated, concentrated and shipped to nearby offshore aquaculture sites while simultaneously remediating coastal waters.

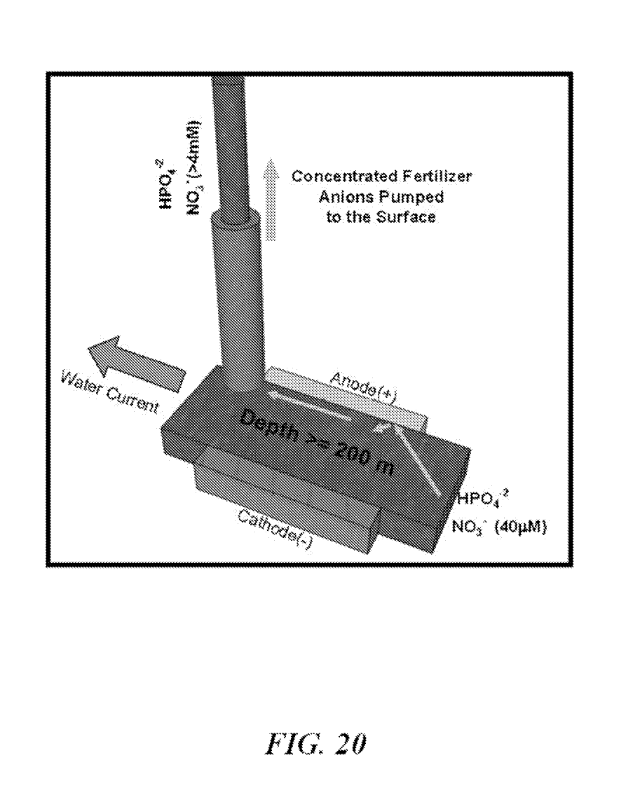

Now that potential deep-water high nitrate and phosphate sources have been identified in areas of low surface concentrations, systems provided herein can access and concentrate these macronutrients based on their ionic charges. If such nutrients are retrieved from the deep ocean, the nutrients may need to be passively concentrated at least 1.5, 2, 3, 4, 5, 6, 7, 8, 9, 10, 20, 30, 40, 50, or 100 times (e.g., 40 uM to 4 mM nitrate) prior to pumping in order to keep the entire process energetically favorable. Nitrate and phosphate anions can be concentrated along the length of the anode by applying a mild current orthogonal to the flow of water, as shown in FIG. 20. The concentrated anion nutrients can be pumped to the surface. The electrical current can be generated via water driven turbine. In some examples, using turbines powered by the winds or currents, the nutrient rich water can be pumped from depths greater than 200 m to a processing platform. Once the nutrient rich water reaches the platform, any nitrates and phosphates in the nutrient rich water can be further concentrated and fed to the cultures as a liquid or precipitated or fed directly to the cultures to generate a hypersaline condition in the vessel, which, for example, can be used as a way to select for a specific species like hypersaline tolerant Nitzschia. The concentrated macronutrients can then be fed to the fertilization vessel in order to achieve macronutrient levels not seen in the open ocean. Accordingly, limiting nutrient concentrations experienced within fertilization vessel may be controlled. In some cases, by closely controlling the vessel's nutrient composition, it can be determined whether or not a species in a biodiverse community will thrive.

Another application of this technology is to remediate coastal estuaries and river deltas by sequestering macronutrient ions before

This disclosure provides vessels to control the cultivation and harvesting of the microalgae induced by manipulating the concentration of the limiting nutrient, which may be iron, nitrate, or phosphate. The vessels may have certain characteristics, such as: 1) to contain microalgae and iron while allowing the hydrophilic nutrients to pass between the vessel and the surrounding environment; 2) to concentrate microalgae prior to harvesting in order to reduce the volume of seawater to be processed and discarded and in turn reduce the energy requirements to process the microalgae; and 3) to maintain or improve environmental conditions.

In some cases, all or substantially all biological growth that is induced by controlling the limiting nutrient is captured. In some examples, organism exposure to limiting nutrient conditions occurs only in the confines of a vessel.

### Vessels

This disclosure provides vessels with features that can improve the microalgae specific growth rates, product density/specificity, and prevent essential nutrients from escaping or being diluted by ocean currents. In some examples, a vessel is adapted such that the exposure of organism to trace metals only occurs in the confines of a vessel. In order to prevent the reduced iron from diffusing outside the vessel, an electromagnetic or permanent magnetic core is provided within the vessel to minimize reduced iron dilution by ocean currents.

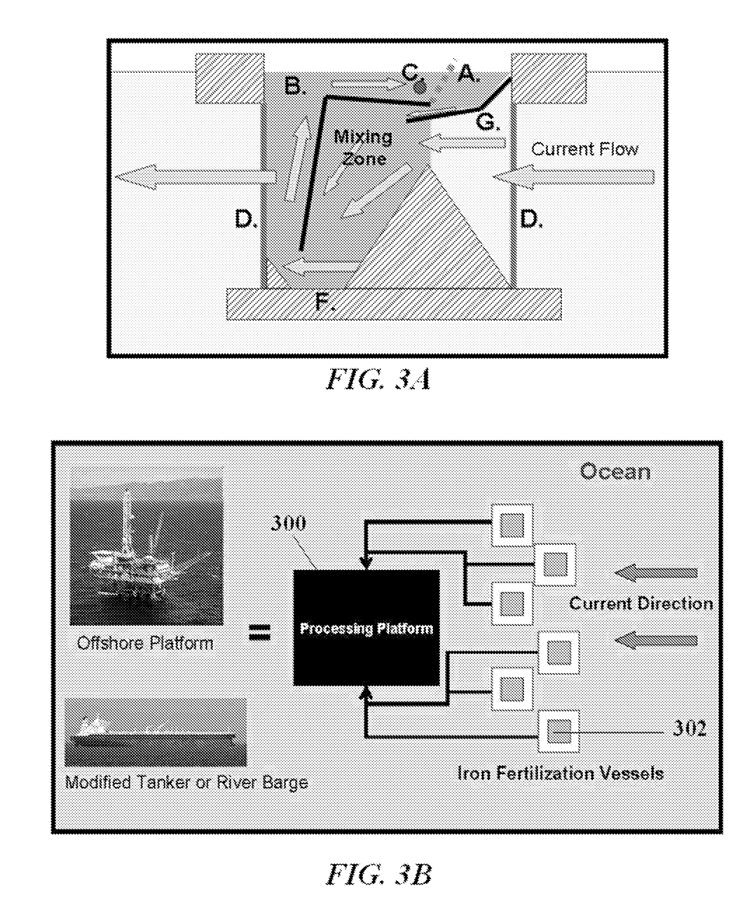

FIG. 3A schematically illustrates a vessel for collecting and concentrating biomass. The vessel of FIG. 3A can be designed to mimic fresh water raceway pond functionality but in a vertical fashion. Increased circulation can allow for better mixing of nutrients and allow for desired or selected exposure of the microalgae to sunlight. The mixing and flow dynamics can be controlled by the design of the vessel dimensions and shape. Baffles within the vessel can be used to control fluid velocity and volumetric flow rates for liquid flowing in a recirculating pattern within the vessel and for liquids flowing through the vessel. The current in the surrounding environment can also be utilized to prevent fouling of the mesh materials by directed current flow across the mesh material or by powering a mechanical cleaning device. In some embodiments, the current can be used to generate power, which can be utilized by the vessel itself in any form, or by the processing platform.

To afford protection from the elements, the vessel, including the semi-permeable walls, can be rigid. In some embodiments of the invention, one wall can be rigid and the other can be flexible. The vessel can be designed to be resistant to damage by weather, current, or any large objects in the surrounding environment. The vessel can be designed to be rigid and protective, while not substantially restricting flow into and out of the vessel from the surrounding environment. In some embodiments, the vessel can be buoyancy controlled to allow the vessel to be submersed during inclement weather. Buoyancy control can be achieved by the top portion, the base, or any combination thereof.

Loss of microorganism and other nutrients through the upstream or current-facing side of the vessel is less of a concern than loss through the down-stream facing portion. In some embodiments, the upstream or current-facing side of the vessel can have a first pore size and the down-stream facing side portion can have pores of a second size that are smaller than the first size.

As shown in FIG. 3A, a permanent magnet that spans the width of the vessel may be incorporated to increase the retentions time that the reduced iron sulfate (C) remains in the upper region of the vessel. The magnet can have a minimum field strength of 6.4 mT. In addition to a component for retaining iron, the vessel may also include components for retaining other nutrients. For example, the vessel may include mechanisms to concentrate nitrates and/or phosphates. The mechanism may include chromatography components, ion-exchange based materials, e.g., ion-exchange columns, and/or affinity based materials, e.g., affinity columns. Any of the vessels described herein may have components for concentration and/or retention of one or more nutrients, e.g., iron, nitrate, and/or phosphate compounds.

Stainless steel sieved gate (shown as dashed lines between C and A in FIG. 3A) with a pore size less than the organism. In some embodiments, the gate is enclosed by the vessel. For example, the gate may be entirely enclosed by the vessel, or at least partially enclosed by the vessel. The gate may be movable between a blocking position and an open position. The gate may be lifted or moved out of a blocking position to an open position to allow the organism to circulate. The gate may be lowered or put in a blocking position to concentrate and harvest the organism (A). FIG. 3A shows the gate (dashed line) in a blocking position.

FIG. 3A indicates a deficient nutrient feed point (B). Feeds that are low in concentration in the surrounding environment can be added at point B. The deficient nutrients that can be fed to the vessel include any nutrient discussed herein. In some embodiments, the nutrients include iron, phosphate, and/or nitrate compounds. The iron can be fed as an iron compound, such as iron sulfate, or iron can be fed to the vessel as part of a biodegradable polymer or material that releases iron over time, as discussed herein. The biodegradable polymer or material can also include other nutrients, such as nitrate compounds and/or phosphate compounds. Nitrates can also be fed in the form of ammonium, ammonium ferrous(II) sulfate (magnetic), or ammonium bicarbonate. Nitrates and other nutrients can also be sourced from waste water, secondary waste water, run off, chicken feed, agricultural waste, or any low-cost nutrient source and then fed to the vessel. The nutrient feed can be controlled automatically or manually. The nutrient feeding may be controlled based on the concentration of the nutrient in the vessel, the growth rate and/or the concentration of the organism. A nutrient feeding component for feeding one or more nutrients can be included in any of the vessels described herein.

The current's kinetic energy can be used to thoroughly mix the micronutrients and the microalgae. The mixing of nutrients and algae can be achieved by baffles within the vessel that direct the fluid in a recirculating pattern. The vessel may be positioned within a flowing current. In FIG. 3A, current flows into the vessel at the right-hand side (D right) and exits the vessel at the left-hand side (D left). The movement from right to left forces circulation within the vessel in the direction indicated by the arrows, which forms a recirculating pattern. The circulation can be created by a Venturi effect caused by the flux of fluid through the reactor from the upstream portion of the vessel to the downstream portion of the vessel. The amount of current flow used for circulation can be selected in a variety of manners, e.g., by altering the exposed surface area on the right hand side of the vessel and/or the surface area on the left-hand side D. In this configuration, the vessel has an upstream, or current-facing side and a downstream or a side that is not facing the current. If the current of the surrounding environment is fixed, the vessel may be fixed in a proper orientation. If the current is not fixed, then the directionality of the vessel may be controlled based on the current's direction. The control of the vessel's orientation can be automatic or manual.

As described elsewhere herein, orientation of the vessel relative to the current in the surrounding environment can plan an important factor in determining the circulation rate within the vessel. To account for this, the vessel can be designed such that the orientation of the vessel with respect to the direction of current flow can be controlled. A self-orienting mechanism capable of orienting the direction of the vessel can be provided. Mechanical features, such as vane-like features, can be used to self-correct or self-orient the direction of the vessel such that a desired flow of water through the vessel is achieved. For example, one or more fin, protrusion, channel, flap, or shaped feature can be provided for the vessel. A self-orienting mechanism can be provided in a stationary position relative to the vessel, or can be movable relative to the vessel.

In some embodiments, the vessel orientation with respect to the current is such that maximal flow through the vessel is achieved. In other embodiments, the vessel orientation can be such that flow through the vessel is lower than the maximal flow through the vessel. For example, if maximal flow is achieved by placing the incoming mesh side the vessel perpendicular to the flow, a lesser amount of flow can be achieved by placing the incoming mesh side at an orientation that is not perpendicular to current flow in the surrounding environment.

The current's kinetic energy can be used to concentrate the microalgae. Once the sieve gate shown in FIG. 3A is placed in a blocking position, the circulation, as described above, can be utilized to concentrate the microalgae against the sieve gate.

All the microalgae spend the same cumulative time in the sun exposure zone (between A and B in FIG. 3A). The amount of time spent exposed to the sun can be controlled based on the circulation rate through the vessel and the cross-sectional area of the channels that allow exposure to the sun relative to the cross-sectional area of the other channels in the vessel.

The recirculation caused by the flux of water through the vessel maintains a constant microalgae density throughout the circulating/recirculating portion of the vessel.

The vessel of FIG. 3A can be designed for high Reynolds number and Peclet number to insure it is in the convection regime for consistent nutrient and organism density.

The pivot point (G) shown in FIG. 3A can control the incoming water velocity. As described above, circulation may be controlled by a variety of manners. Here, an incoming water gate can control or restrict the rate of water entering the vessel.

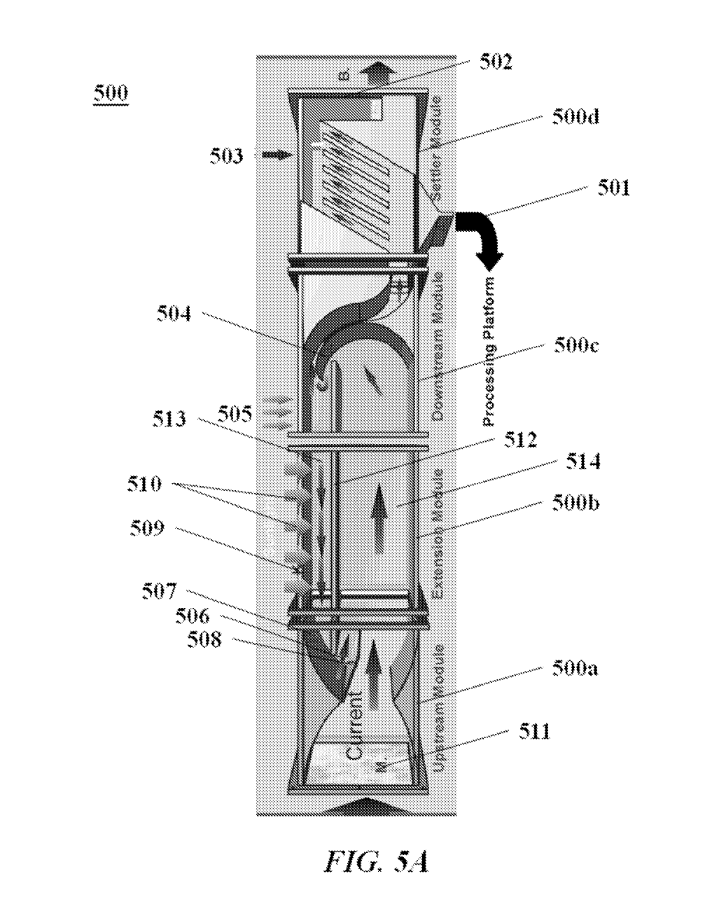

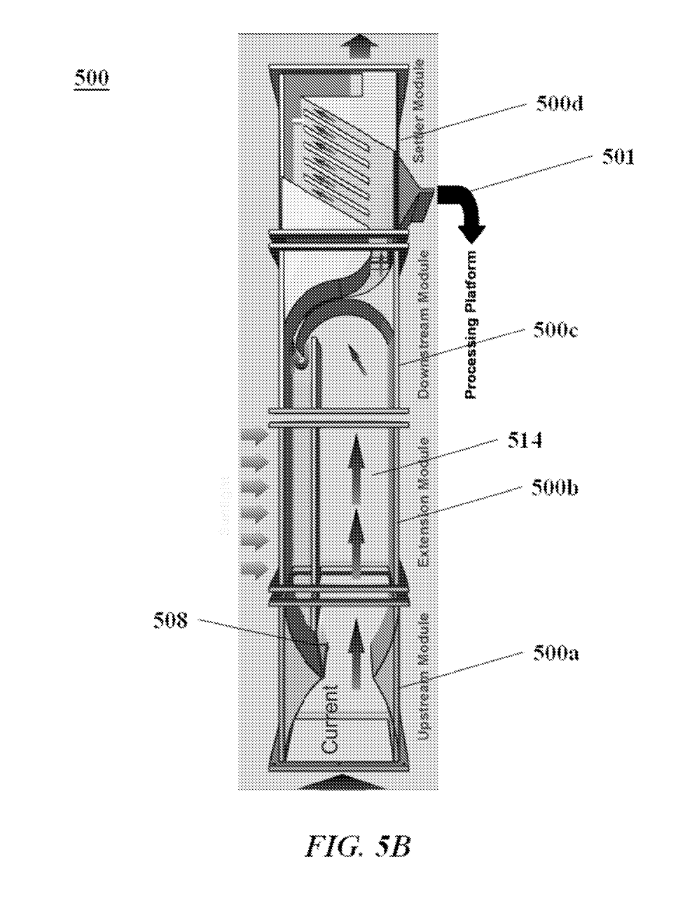

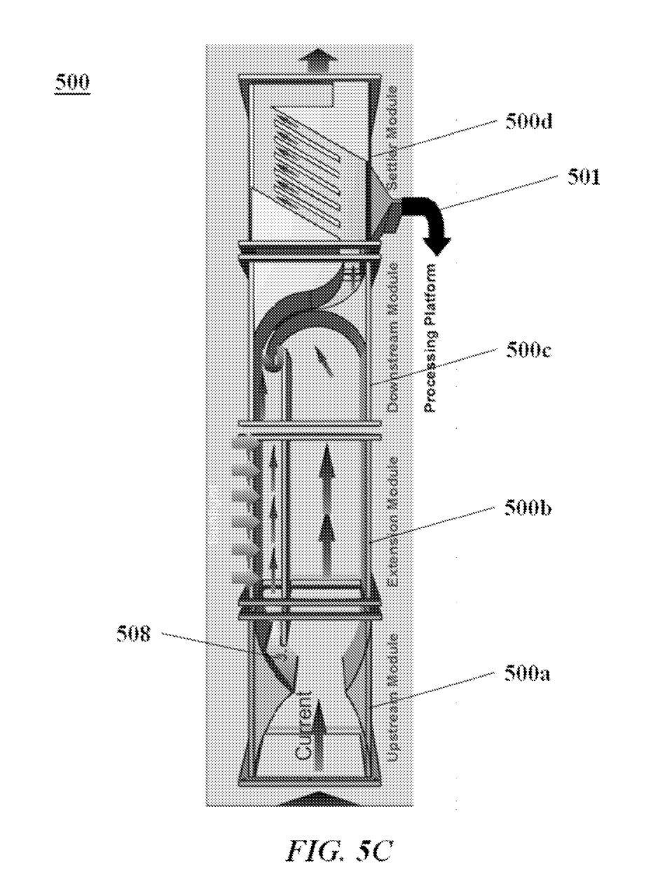

If necessary, the vessel percolates or sparges carbon dioxide from the base (F) shown in FIG. 5 in an effort to achieve higher microalgae densities.

A hydrophilic, charged, porous material (D) shown in FIG. 3A can allow environmental micronutrients and waste organic acids to cross freely but contain the microalgae. This can be achieved by selecting an appropriate pore size, e.g., less than about 5, 10, 15, 20, 30, 50, 100, or 150 μm pore size (or any other pore size described herein).

FIG. 3B displays a possible layout around an out of service drilling platform 300. The platform offers a place to harvest and process the microalgae on site into renewable hydrocarbon fuels, protein supplements, and glycerol which is preferred because it is more efficient to transport liquid fuels. As shown in FIG. 3B, a tanker or river barge retrofitted with the tools necessary for doing the microalgae to renewable fuels conversion at sea is also a viable option. In some embodiments, one or more vessels 302 may be provided upstream of or in proximity to the platform 300.

Existing open-water enclosures utilize mesh netting with pore sizes less than 30 μm. These mechanisms can allow continuous nutrient replenishment through the mixing by ocean currents, which captures the microalgae product. They often incorporate a large buoyant top to support the structure.

This disclosure provides vessels that may be modular. In some cases, a vessel is a single unit. In other cases, a vessel includes a plurality of modules. The modules can be separable from one another. Such modules can be fastened or otherwise secured to one another with the aid of fastening members (e.g., screws, bolts, wires, welds, glue). As an alternative, the modules are not separable from one another. A vessel can include a plurality of modules, at least some of which can at least partially define a fluid flow path or a plurality of fluid flow paths of the vessel.

In some cases, a system for collecting and/or harvesting biomass can include a first module adapted to accept a fluid stream and a second module downstream of the first module. The first module includes a gate that is adapted to regulate the flow of the fluid stream along a fluid flow path leading from the first module to the second module. The fluid flow path can be part of a circulatory fluid flow system. The second module accepts a fluid from the first module and directs at least a portion of the fluid to the first module. The system can further include a third module downstream of the second module. The third module includes one or more surfaces for retaining one or more microorganisms upon the flow of at least a portion of the fluid stream through the third module.

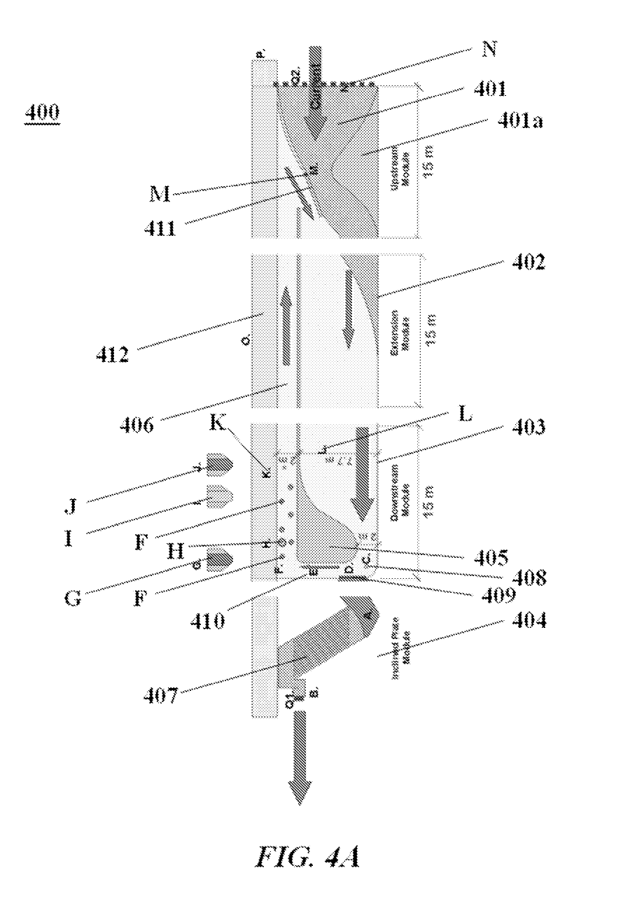

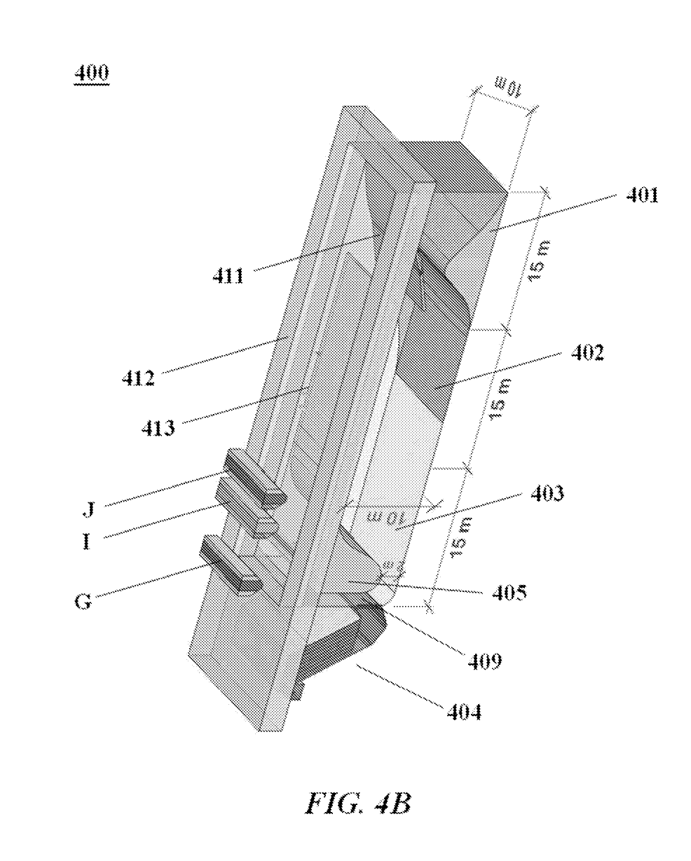

FIGS. 4A and 4B show a vessel 400 adapted to harvest biomass. The vessel 400 includes a plurality of modules: an upstream module 401, an extension module 402, a downstream module 403 and an inclined plate module (or settler module) 404. The vessel 400 includes a circulatory fluid flow path directed through the upstream module 401, extension module 402 and downstream module 403 in a circulatory fashion. The modules 401-404 may be connected to adjacent modules with the aid of mechanical fasteners, such as welding, bolts, or screws. The vessel 400 is configured to be submerged in a body of water to a given depth. During use, a current (arrows) enters the upstream module 401 and is directed to the downstream module 403 through the extension module 402. Current in the downstream module 403 is directed around a divider 405 and is directed through a passageway 406. In some cases, the extension module 402 is precluded.

The upstream module 401, extension module 402 and downstream module 403 can have various lengths and shapes. In an example, the modules have lengths of about 15 m and widths of about 10 m.

The upstream module 401 can include an incline 401a to shape or otherwise influence characteristics of current entering the upstream module 401. The incline 401a can be have a concave shape along a direction leading from an inlet of the upstream module 401 into the vessel 400.

The vessel 400 can include a pump that delivers concentrated biomass back to the vessel 400 during the growth phase or to the next growth vessel or processing platform. The vessel 400 includes magnetic members F, which can be magnetic rods or electromagnets. In the illustrated examples, the magnetic members F are situated in the downstream module 403. Iron that passes the magnetic members can be collected with the biomass.

A portion of the current (or fluid) can be directed to the incline plate module 404. The incline plate module 404 can include one or more plates 407 to permit algae or other biomass to settle during fluid flow through the incline plate module 404. The incline plate module 404 can include a hydrophobic membrane, which can prevent algae that have not settled from escaping the vessel 400. The plates may be as described in PCT Patent Publication No. WO/2010/141559, which is entirely incorporated herein by reference. The plates can be formed of corrosion resistant and/or polished metal, such as, for example, stainless steel, aluminum, coated mild steel, a polymeric material or combinations thereof. The incline plate module 404 may include a retaining mesh at an exit of the module 404. In some examples, the retaining mesh can be formed of plankton netting.

The vessel can include probes 408 that measure nutrient (e.g., nitrate, phosphate, and iron) concentrations. The exit concentration values can determine how much of these nutrients are fed at points G (iron), I (nitrate), and J (phosphate). The vessel 400 can include a probe that that measures the water velocity, optical density, and Chlorophyll a concentrations.

The vessel 400 includes a waste gate 409 that can control how much water recycles or exits the vessel 400. The gate 409 can have a metering feature. In some example, the metering feature will allow one to control the percentage of water that is recycled. At low culture densities, this gate will be closer to the closed position to force more of the media to recycle. As the culture grows in the incline plate module 404, the gate 409 can open further to allow the media to be replenished faster.

The gate 409 can function by opening or constricting a passageway leading to a gap 410. The gap 410 can elevate the water velocity enough to entrain the biomass and transport it to the top of the downstream module 403 and to the passageway 406. The gate 409 can regulate fluid flow through the inclined plate module 404. For instance, the gate 409 can be in a vertical position to close an opening into the incline plate module 404 but permit fluid flow through the gap 410. As another example, the gate 409 can be in a non-vertical (e.g., horizontal) position, thereby permitting fluid flow through the inclined module 404.

The upstream module 401 can include an upstream gate 411. The gate 409 in conjunction with the upstream gate 411 can control the depth of water in upstream module 401 module, extension module 402 and downstream module 403.

The biomass may require light for growth. In some examples, as the density of the biomass increases, the upper level depth can be decreased in order to allow light to reach the bulk of the biomass.

The vessel 400 can include one or more magnetic members F that span the width of the vessel 400 in order to increase the retention time of iron (e.g., Fe²⁺) in the upper region of the vessel, such as in the passageway 406. The magnetic members can be in the upstream module 401, extension module 402 and/or downstream module 403. A magnetic member can have a field strengths of at least about 1 mT, 2 mT, 3 mT, 4 mT, 5 mT, 6 mT, 7 mT, 8 mT, 9 mT, 10 mT, 20 mT, 30 mT, 40 mT, 50 mT, 100 mT, or 1000 mT. In some examples, a magnetic member has field strength of at least about 6.4 mT.

The concentration of nutrients in the vessel 400 can be regulated. An iron source G permits the introduction of concentrated iron (II) nutrients. Concentrated phosphate can be introduced at source I. Concentrated nitrate can be introduced at point J. The introduced nutrients can traverse the entire vessel pathway (e.g., passageway 406), including a mixing turbulent region of the vessel 400. In some cases, the vessel 400 can include a pipe H for providing unprocessed nutrient rich deep water into the vessel 400. The vessel 400 can include probes K that measure water temperature, water velocity, water salinity, and/or chlorophyll concentration. The probes K can be situated in the downstream module 403, though other locations and/or configurations are possible. The turbulent region is at location M in FIG. 4A. At the turbulent region, the venturi effect may pull a fluid from the passageway 406 into a fluid stream directed into the vessel 400 at point N in the upstream module 401. The incoming flow should be sufficient to mix the fresh incoming flow with the culture from the upper chamber.

In some situations, a height of the vessel L, such as the height of the downstream module 403, can be decreased during the vessel's fabrication to decrease the cross-sectional area of the downstream module. If the height at point N (i.e., vessel height specification at water inlet) remains the same, decreasing the height at point L can increase the maximum achievable water velocity through the vessel.

The gates 409 and 411 can be electric gates or hinged gates. In some situations, the gates 409 and 411 are hinged gates, adapted to open and close upon the application of a force across a surface of the gate. For instance, fluid flow through the upstream module 401 can cause the gate 411 to open and close based on the flow rate of fluid through the upstream module 401. The gates can control the velocity and volume of the inlet water supply.

The vessel 400 can include a lid 412 that allows the vessel 400 to retain secreted oils that can be trapped against the lid due to density differences between the water and the oils. The oils can be siphoned off as they accumulate. In addition, the lid 412 can have an optical window 413 to allow the entire spectrum or specific wavelengths of light to enter/transmit into the vessel 400. Controlling the light spectrum that enters the vessel can be another way to select for specific biomass, such as algae species.

The vessel 400 can include a system for buoyancy control. Buoyancy control can allow the depth at which the vessel 400 operates to be controlled or regulated. Having the ability to submerge the vessel 400 without losing containment may minimize damage during rough seas since surface energy minimizes with depth.

The vessel 400, including the modules 401-404, may be formed of a metallic, semiconductor, insulating, polymeric, ceramic, or composite material. In an example, the vessel 400 is formed of a polymeric material, such as plastic. In another example, the vessel 400 is formed of a biodegradable material. In another example, the vessel 400 is formed of a low embodied energy material.

The vessel 400 can include one or more membranes/sieves at the entrance of the vessel 400 (at the upstream module 401) and at the exit of the vessel 400 (at the inclined plate module 404) to allow water soluble nutrients to be replenished and the generated organic acids to be flushed from the system through its pores. At the same time, the membranes/sieves can retain the selected organism in the vessel while minimizing contamination by external species of algae and zooplankton.

In some situations, however, the use of a membrane or sieve at the entrance of the vessel, such as at an entrance of the upstream module 401, may restrict the flow of water into the vessel 400 and prevent or impede the circulation of the algae culture within the vessel 400. This problem can be minimized or eliminated by using forced induction.

Forced induction may work best in very strong currents, such as in the Gulf Stream. Strong currents may be required because some of the energy is dissipated during the transmission from the external fluids to the internal fluids. This may be analogous to that of a turbo charger in a car that uses the exhaust gas to drive the internal intake induction propeller. The fertilization vessel's possible use of forced induction requires a way to use the external energy in the current to drive the circulation in the vessel and overcome the flow impedance caused by the inlet filtration membrane/sieve.

Forced induction can be implemented with the aid of one or more impellers. In some examples, an impeller is a rotor inside a tube or conduit used to increase (or decrease in case of turbines) the pressure and flow of a fluid.

In some cases, the vessel 400 can include an external impeller and an internal impeller. The external impeller can be disposed outside of the vessel, including the modules 401-404 of the vessel, while the internal impeller can be disposed inside the vessel 400. In an example, the vessel includes an impeller module upstream of the upstream module 401. The impeller module includes the external impeller. The internal impeller is situated in the upstream module 401. The upstream module and the impeller module can be fluidically isolated from one another, or, as an alternative, the flow from the impeller module to the upstream module 401 may be restricted. As another alternative, the membrane/sieve may be situated between the impeller module and the upstream module 401.

The external impeller can be coupled to the internal impeller. The external impeller can be driven by the current, which in turn can drive the internal impeller to generate or facilitate fluid flow in the vessel 400. The internal impeller can create a pressure gradient across the membrane/sieve to force the water to enter the vessel 400.

Biomass can be collected using vessels provided herein, such as the vessel 400 of FIGS. 4A and 4B. Biomass can be harvested by directing the mature culture through the inclined plate clarifier (e.g., inclined plate module 404 of FIG. 4A) by manipulating a gate leading into the inclined plate clarifier (e.g., gate 409) and pumping the settled biomass to the processing platform.

FIGS. 5A-5C schematically illustrate a vessel 500 configured for collecting biomass. The vessel 500 includes an upstream module 500a, extension module 500ab, downstream module 500c and settler module 500d. The settler module (or inclined module) 500d includes a plurality of plates for enabling biomass to settle thereon upon fluid flow through the settler module. The direction of fluid flow is shown in the figures by arrows. The modules of the vessel 500 can be similar to that of a train boxcar, such as, for example, 60′×10′7″×10′7″.

During operation of the vessel, biomass is collected in the settler module. A pump 501 delivers concentrated biomass collected in the vessel 500 during the growth phase back to the vessel 500 or to another growth vessel or processing platform. The settler module includes a hydrophobic membrane 502 that prevents free flowing biomass (e.g., algae) from escaping the vessel 500. The vessel 500 includes probe(s) 503 that measure the nitrate, phosphate, and iron nutrient concentrations. The exit concentration values can determine how much of these nutrients are fed into the vessel 500. There is also a probe that measures the optical density, pH and Chlorophyll a concentrations for exit water quality purposes.

The vessel 500 can include a small gap 504 that elevates the water velocity enough to entrain the biomass and transport it to the top of the vessel and pass the exit. We are relying on inertia and gravity to prevent the induced biomass growth from making the 180 degree change in direction needed to enter the inclined plate settler module. The vessel 500 includes a point 505 at which concentrated iron (II) nutrients are introduced and allowed to traverse the entire vessel pathway including in the mixing turbulent region. At point 505, concentrated nutrient-rich (nitrate, phosphate, and bicarbonate) deep ocean water can be fed into the vessel 500 based on the continuous feedback of probe(s) 503 and the optical density of the culture. In some cases, at point 505 concentrated imported phosphate can be introduced and allowed to traverse an entire pathway of the vessel 500 pathway, including in a mixing turbulent venturi region near point 506. In some cases, concentrated imported nitrate can be introduced and allowed to traverse the entire pathway of the vessel 500, including in the mixing turbulent venturi region near point 506.

The vessel 500 can include probers at point 507 that measure temperature, water velocity, salinity, and chlorophyll concentration. The vessel 500 can include a hinged gate 508 that controls the velocity and volume of the inlet water supply during the growth and harvesting phase. The illustrated position is used during specifically for the growth phase. Two harvesting position(s) of the gate 508 is discussed elsewhere herein.

The vessel can include a sealed lid 509 that allows the vessel 500 to retain secreted oils trapped in designed pockets in the lid due to density differences between the water and the oils. The oils can be siphoned off as they accumulate. In addition, the lid can allow sunlight 510 to penetrate into a euphotic zone of the vessel 500. The lid can be tinted to allow the entire spectrum or specific wavelengths of light to enter/transmit into the vessel. Controlling the light spectrum that enters the vessel is another way to select for given algae species.

The vessel 500 can include buoyancy control to regulate the depth at which the vessel 500 operates. Having the ability to submerge the vessel without losing containment minimizes damage during rough seas since surface energy can be reduced with depth.

Membranes/sieves 511 can be disposed at the entrance of the vessel 500 (e.g., at the upstream module 500a) to allow water soluble nutrients to be replenished and the generated organic acids to be flushed from the system through its pores. The vessel 500 can include membranes/sieves at an exit of the vessel 500 (e.g., at the exit of the settler module 500d). The membranes/sieves can retain the selected organism in the vessel while minimizing/delaying contamination by external species of algae and zooplankton. The use of a membrane/sieve can restrict the flow of water into the vessel 500 and prevent the circulation of the biomass (e.g., algae culture) within the vessel 500. Using the forced induction described elsewhere herein can aid in preventing the circulating of the biomass culture in the vessel 500.

An issue with membrane/sieves which may arise in some cases is bio-fouling. Membrane/sieve bio-fouling can include clogging of the membrane pores with environmental debris. Bio-fouling can be mitigated when a membrane/sieve is at least partially formed of or coated by an anti-biofouling additive selected from the group consisting of polyethylene glycol (PEG), hyperbranched fluoropolymer (HBFP), polyethylene (PE), polyvinyl chloride (PVC), polymethylmethacrylate (PMMA), natural rubber (NR), polydimethylsiloxane (PDMS), polystyrene (PS), perfluoropolyether (PFPE), polytetrafluoroethylene (PTFE), and silicons and derivatives.

Bomass can be harvested by directing a mature culture through an inclined plate clarifier in the settler module 500d by manipulating the gate 508 and pumping the settled biomass to the processing platform with the aid of the pump 501. The harvesting mechanism can have two stages. In a first stage shown in FIG. 5B, the gate 508 is in line with a partition 512. The partition 512 divides chamber 513 and a lower chamber 514. This directs fluid flow through the lower chamber 514, which evacuates the lower chamber 514. After the lower chamber 514 is cleared, the system can (1) continue in the growth phase by returning the gate to its original position illustrated in FIG. 5A or (2) harvest the upper chamber by raising the gate 508 to position further to the position illustrated in FIG. 5C, which permits fluid flow into the upper chamber 513. Aluminum sulfate and/or magnetite can be directly added to the settler module 500d in order to induce aggregation as the culture is harvested, as described in PCT Publication No. WO/2010/141559 and PCT Application No. PCT/US2012/041766, each of which is entirely incorporated herein by reference. The settled biomass sludge can then be pumped 501 to a processing platform. The use of magnetite can allow the biomass to be concentrated by a magnetic field, which can significantly decrease the dewatering energetic costs.

The vessel 500 can only include a single gate. Flow metering can be implemented, for instance, with the aid of the gate 508 of FIGS. 5A-5C.

In some case, a collector of biomass can include a vessel comprising (i) one or more surfaces for collecting a biomass from a fluid directed through the vessel and (ii) one or more internal impellers in fluid communication with the one or more surfaces through a fluid flow path. The one or more internal impellers can facilitate flow of the fluid through the fluid flow path. The fluid flow path can be part of a circulatory fluid flow system of the vessel. The collector can include an external impeller coupled to the one or more internal impellers. The external impeller can be disposed external to the housing and adapted to provide rotational energy to the internal impeller upon fluid flow through or adjacent to the external impeller.

The external impeller and or internal impeller may be regulated by a control system having a processor that is programmed to measure and/or regulate one or more parameters of the external impeller and/or internal impeller, such as rate of rotation, and whether an impeller is permitted to rotate (e.g., whether a braking mechanism of the impeller is engaged, thereby preventing rotation, or disengaged, thereby permitting rotation). The control system can aid in regulating the flow rate of fluid in the collector (or vessel).

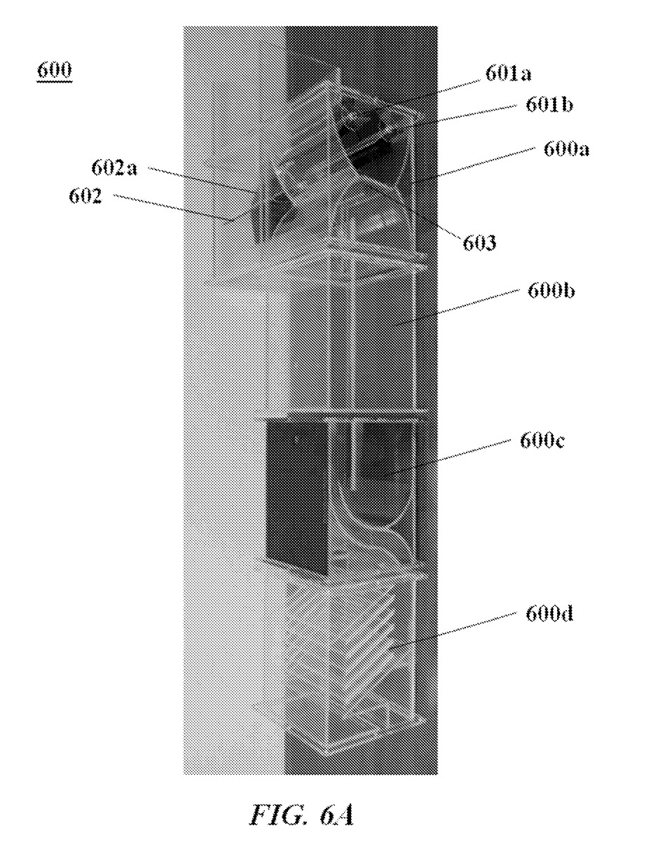

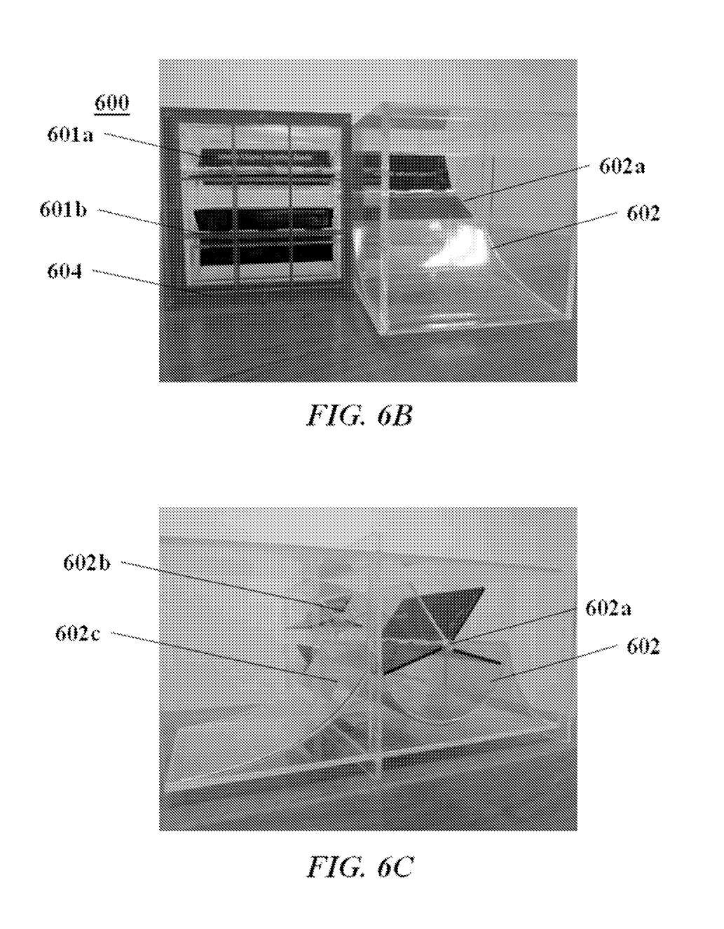

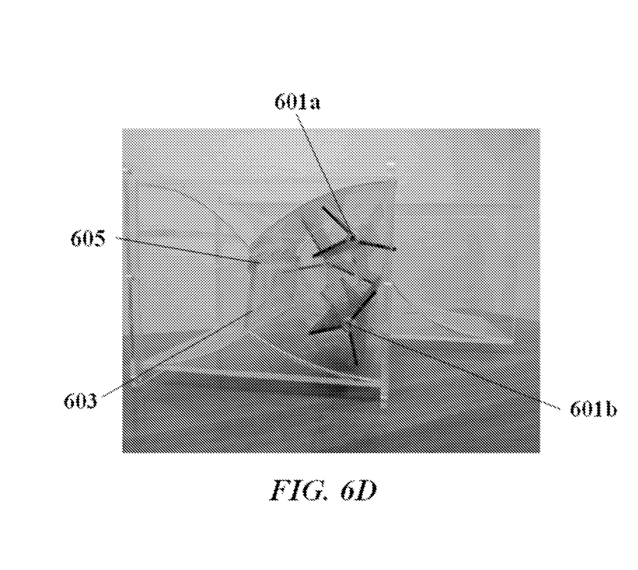

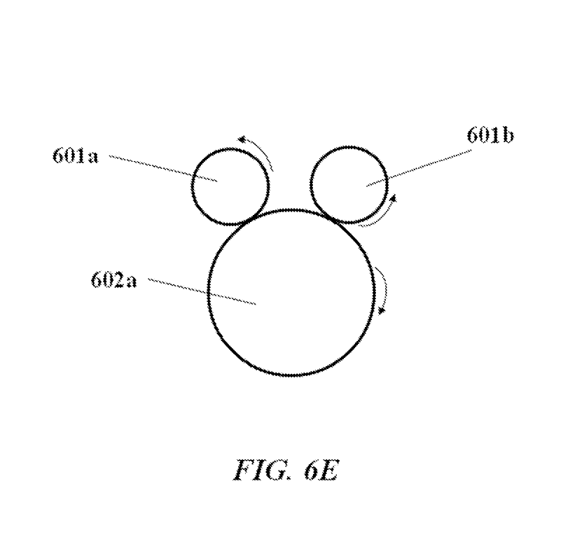

Fluid circulation in vessels of the disclosure can be facilitated with the aid of forced induction. FIGS. 6A-6D show an example of a vessel 600 with a forced induction module 600a. The vessel 600 includes the forced induction module 600a, extension module 600b, downstream module 600c and settler module 600d. The forced induction module 600a includes a first internal impeller 601a and a second internal impeller 601b, which can be operatively coupled to one another through a gear (e.g., side gear with interlocking spokes to couple rotational motion). The vessel 600 includes an external impeller module 602 with an external impeller 602a. The external impeller 602a can be operatively coupled to the internal impellers 601a and 601b through a gear mechanism, such as gears with interlocking spokes. The forced induction module 600a includes a metering gate 603, which can be a hinged gate. The metering gate 603 can regulate fluid flow into the extension module 600b.

With reference to FIG. 6B, the forced induction module 600a includes a membrane/sieve holder 604. In an example, the forced induction module 600a includes one or more openings (e.g., slots, slits, etc.) to permit fluid to enter the forced induction module 600a. Rotational energy provided through the external impeller 602a can facilitate fluid flow and circulation in the vessel. The one or more openings can be disposed such that they are in direct contact with a flowing current, or can be situated at locations that are not in direct contact with a flowing current. In another example, the forced induction module 600a does not include any openings to the external environment. The forced induction module 600a in such a case can include a side opening that is fluidically coupled to the external impeller module 602, and fluid is directed into the forced induction module 600a through the external impeller module 602.

The external impeller module 602 can include an opening 602b and a ramp 602c, as shown in FIG. 6C. The opening 602b and ramp 602c can be adapted to face current flow. In an example, during use, a fluid can directly impinge the ramp 602c and be directed to the opening 602b.

With reference to FIG. 6D, the metering gate 603 can have a hinge 605 that is adapted to enable the gate 603 to pivot. Pivoting of the gate 603 can enable the gate to withdraw energy from the flowing fluid, thereby aiding in regulating fluid flow. In some situations, the gate 603 can be electrically controlled to regulate fluid flow through the vessel 600. For instance, the gate 603 can include a locking member that locks the gate 603, thereby impeding fluid flow from the forced induction module 600a to the extension module 600b.

An impeller can include a rotor and one or more blades extending outwardly from the rotor.

FIG. 6E shows the interlocking gears of the external impeller 602a and internal impellers 601a and 601b (spokes not shown). The arrows indicate example directions of rotations of the impellers. As an alternative, the external impeller 602a can be coupled to one (but not both) of the internal impellers 601a and 601b, and one of the internal impellers 601a and 601b can be coupled to the other of the internal impellers 601a and 601b.

The external impeller module 602 can include at least 1, 2, 3, 4, 5, 6, 7, 8, 9, 10, 20, 30, 40, 50, or 100 external impellers. The forced induction module 600a can include at least 1, 2, 3, 4, 5, 6, 7, 8, 9, 10, 20, 30, 40, 50, or 100 internal impellers.

During use of the vessel 600, current (e.g., sea current, deep water current) flows through the external impeller module 602 and impinges on one or more blades of the external impeller 602a, which causes rotation of the external impeller 602a. The external impeller is mechanically coupled to the internal impellers 601a and 601b. Upon rotation of the external impeller 602a, the internal impellers 601a and 601b can also rotate. Rotation of the internal impellers 601a and 601b generates fluid motion in the forced induction module 600a, which drives fluid flow in the vessel 600 by forced induction.

### Nutrient Concentration

This disclosure provides systems and methods for concentrating nutrients that can promote the growth of microorganisms, herein also “fertilizers.” Nutrients may be concentrated and/or recycled using a magneto hydro fertilizer concentrator (MHFC), operating on the principle of magnetohydrodynamics. In some cases, nutrient concentrators are coupled to vessels for collecting and/or growing biomass, such as vessels described elsewhere herein (see, e.g., FIGS. 4-6).

Faraday's Law describes how a time varying magnetic field creates (“induces”) an electric field. According to Faraday's Law, an electromotive force (EMF) is generated when an electrolytic stream flows through a magnetic field. This EMF drives the ions through cation and anion-selective membrane, thus either concentrating or diluting salt concentrations in this way. This method may be considered as a self-powered electrodialysis unit. The EMF generated is proportional to the rate of change of the magnetic flux. The variables that may significantly impact the magnetic flux in the MHFC are (i) the electrolyte concentration, (ii) the fluid velocity, and (iii) the magnetic field strength.

An MHFC can amplify seawater current velocities by reducing the flow cross-sectional area. Increasing the electrolyte fluid velocity can proportionally increase the electromotive force within the electrodialysis stack. The MHFC can constantly produce an electrical current that can simultaneously concentrate fertilizer ions in the electrodialysis stack and powers equipment like pumps. Moreover, the essential nutrients derived from the MHFC can be fed into the vessel. In addition, the net energy consumption and operational costs inputs are well below those experienced by Reverse Osmosis (RO).

MHFC's of the disclosure can advantageously have a relatively simple flow through design with practically no moving parts, and can manage/minimize fluid processing volumes. This is in contrast to at least some RO systems, which may require relatively large volumes of nutrient rich waters to be pumped to the surface and processed.

An MHFC can concentrate ocean fertilizer at any depth that is abundant with nutrients. Since the MHFC concentrates the ocean fertilizer at depth, it minimizes the volume of water that must be pumped to the surface. In some cases, using an array of rare earth permanent magnets in order to generate the current that drives the electrodialysis can minimize the external energy input(s) of the nutrient concentrator. In addition, the concentrated nutrients may not be pumped continuously but intermittently when the nitrate in the concentrator chambers exceed a minimum value, thus minimizing pumping volume and energy requirements for the concentrator. Concentrators of the disclosure can have various shapes, sizes and configurations. In some cases, a concentrator can be the size of a railroad car or truck trailer (e.g., having a characteristic dimension, such as a length, of about 50-100 feet) in order to facilitate its transport from the manufacturer to the end user, or it can be smaller, such as the size of a room (e.g., having a characteristic dimension of about 10-20 feet), or even smaller, such as the size of a cabinet (e.g., having a characteristic dimension of about 1-2 feet) that can fit on top of a regular-sized desk. Ion separation using magnetohydrodynamics is described in, for example, U.S. Pat. No. 6,768,109 to Brokaw et al. and U.S. Pat. No. 7,033,478 to Harde, which are entirely incorporated herein by reference. Nutrients may be concentrated directly in vessels described herein. In some cases, the vessels may be adapted with one or more devices that allow for concentration of nutrients from the ambient environment, such as an aquatic environment, and direct the nutrients into a region of the vessel, such as an enclosure, where microorganisms, such as algae, may grow.

A vessel can include a semi-permeable enclosure and a magnetic field source outside (or external to) the semi-permeable enclosure. The vessel can be as described elsewhere herein, such as any of the vessels of FIGS. 4-6. The enclosure can retain a microorganism. The magnetic field source is capable of inducing a magnetic force that directs one or more water-soluble nutrients in a fluid current into the semi-permeable enclosure. The vessel can be located in an aquatic environment, such as an ocean. In such cases, the fluid in the fluid current may be, without limitation, seawater, river water, or lake water. The vessel can further include a sieved gate, which can be partially enclosed by the semi-permeable enclosure. The gate can be movable between a blocking position and an open position. The gate can be formed of stainless steel or other materials. An example of sieved gate is shown in FIG. 3A (shown as dashed lines between C and A).

The vessel can also include a self-orienting mechanism capable of orienting the direction of the vessel with respect to the direction of the fluid current flow when the vessel is positioned in the fluid current. Mechanical features, such as vane-like features, can be used to self-correct or self-orient the direction of the vessel such that a desired flow of water through the vessel is achieved. For example, one or more fin, protrusion, channel, flap, or shaped feature can be provided for the vessel. A self-orienting mechanism can be provided in a stationary position relative to the vessel, or can be movable relative to the vessel.

The vessel can include a mechanism for directing water-soluble nutrients into the semi-permeable enclosure. In some embodiments, the mechanism can be a pipe, a pump, a channel, or a passageway for conveying the nutrients, whose concentration in the fluid may be increased with the aid of a magnetic field source, into the semi-permeable enclosure. The nutrients being directed or conveyed into the enclosure can be fully dissolved in the fluid, or they can be partially precipitated, forming a slurry or suspension with the fluid. In some cases, the fluid can be seawater, such as ocean water. In other cases, the fluid can be fresh water, such as river water.

The magnetic field source can increase the concentration of the nutrients in the fluid relative to the fluid untreated with a magnetic field source by a factor of at least 1.1, or at least 1.2, or at least 1.3, or at least 1.4, or at least 1.5, or at least 2, or at least 2.5 or at least 3, or at least 3.5, or at least 4, or at least 4.5, or at least 5, or at least 6, or at least 7, or at least 8, or at least 9, or at least 10, or at least 15, or at least 20, or at least 25, or at least 30, or at least 40, or at least 50, or at least 100. In some cases, the concentration of one or more nutrients can exceed the solubility limit of the nutrient in the fluid, such that precipitation can occur. In those cases, the one or more nutrients can form a slurry or a suspension with the fluid. In those cases, the pipe, pump, channel, or passageway for conveying the nutrients can be large enough (e.g., can have a large enough diameter) such that the nutrients can be conveyed into the semi-permeable enclosure without clogging or otherwise blocking the pipe, pump, channel, or passageway. In other cases, the pipe, pump, channel, or passageway can include a filter or a membrane such that precipitated or undissolved materials in the fluid can be removed. In those cases, the fluid can be saturated with water-soluble nutrients by the time it reaches the semi-permeable enclosure.

The concentration of a nutrient in a fluid, after the fluid including the nutrient passes through an area under the influence of a magnetic field source, can increase relative to the concentration of the nutrient in the fluid that did not pass through an area under the influence of a magnetic field source. In some embodiments, the concentration of a nutrient in the fluid can increase to 0.0001 mol/L (M), or 0.0005 M, or 0.001 M, or 0.005 M, or 0.01 M, or 0.05 M, or 0.1 M, or 0.5 M, or 1 M, or 1.5 M, or 2 M, or 3 M, or 4 M, or 5 M, or 6 M, or 7 M, or 8 M, or 9 M, or 10 M after the fluid including the nutrient passes through the area under the influence of a magnetic field source. After the fluid including the nutrient enters the area under the influence of a magnetic field source, the fluid can be separated into two or more streams. One of the streams can include the fluid with a higher concentration of the nutrient than the fluid before it reached the area under the influence of a magnetic field source, while another stream can include the fluid with a lower concentration of the nutrient than the fluid before it reached the area under the influence of a magnetic field source. In some cases, the two or more streams can be separated. The stream including the nutrient with an increased concentration can be conveyed into the semi-permeable enclosure, while the stream including the nutrient with a reduced concentration can be conveyed back into an area outside the vessel, such as an aquatic environment.

The vessel can include a mechanism for increasing the velocity of the fluid current. The fluid current, such as a current of the flow of seawater or river water, can have its own natural velocity. The mechanism can increase the velocity by, for example, reducing the flow cross-sectional area, such as by a narrowing passageway, or channel, that focuses the flow of current from the ambient environment into the vessel, including the part of the vessel that can be under the influence of a magnetic field source. The channel can increase the velocity of the natural current by a factor of at least 1.1, or at least 1.2, or at least 1.3, or at least 1.4, or at least 1.5, or at least 2, or at least 2.5 or at least 3, or at least 3.5, or at least 4, or at least 4.5, or at least 5, or at least 6, or at least 7, or at least 8, or at least 9, or at least 10, or at least 15, or at least 20, or at least 25, or at least 30, or at least 40, or at least 50, or at least 100. The velocity of the current, after the current undergoes an increase in the velocity in the passageway or channel, can be at least 0.5 m/s, or at least 1 m/s, or at least 1.5 m/s, or at least 2 m/s, or at least 2.5 m/s, or at least 3 m/s, or at least 3.5 m/s, or at least 4 m/s, or at least 4.5 m/s, or at least 5 m/s, or at least 5.5 m/s, or at least 6.5 m/s, or at least 7 m/s, or at least 8 m/s, or at least 8.5 m/s, or at least 9 m/s, or at least 9.5 m/s, or at least 10 m/s, or at least 12 m/s, or at least 15 m/s, or at least 20 m/s, or at least 25 m/s, or at least 30 m/s, or at least 40 m/s, or at least 50 m/s.

The water-soluble nutrients can include electrolytes. Electrolytes in some cases can include free, or dissociated, ions in a solution. The ions can be dissolved in water or other fluid. In some cases, the electrolytes can be in the form dissolved salts, but they can also be solutions of acids and bases. Electrolytes can make the substance in which they are dissolved electrically conductive.

Electrolytes described herein can include various ionic, acidic, or basic substances. Among ionic substances, some can include cations such as lithium, sodium, potassium, rubidium, cesium, magnesium, calcium, strontium, barium, scandium, yttrium, titanium, zirconium, vanadium, niobium, tantalum, chromium, magnesium, tungsten, manganese, rhenium, iron, ruthenium, cobalt, rhodium, iridium nickel, palladium, platinum, copper, silver, gold, zinc, aluminum, gallium ammonium, phosphonium, and other cations. These cations can have the charge of +1, +2, +3, +4, +5, +6, or +7. Among ionic substances, some can include anions such as fluoride, chloride, bromide, iodide, oxide, sulfide, nitride, carbonate, nitrate, nitrate, phosphate, phosphite, tungstate, molybdate, chlorite, chlorate, bromite, bromate, acetate, sulfite, sulfate, hydrogen carbonate, hydrogen phosphate, silicate, borate, aluminate, cyanide, thioscyanate, hydroxide, permanganate, oxalate, vanadate, chromate, and dichromate. These anions can have the charge of −1, −2, −3, −4, −5, −6, or −7. In a fluid solution, the cations and anions are separated from one another and are typically surrounded by molecules of the fluid, such as water. In an undissolved form, these cations and anions can be combined to form salts, such magnesium chloride, potassium nitrate, calcium carbonate, sodium phosphate, calcium bromide, silver oxalate, copper chloride, nickel phosphate, zinc iodide, ammonium chloride, tetrabutylammonium bromide, and barium silicate, where the cations and anions are bound to each other via ionic bonds, sometimes in an ionic lattice.

Electrolytes described herein can also include acids, such as acetic acid, phosphoric acid, phosphorous acid, carbonic acid, hydrochloric acid, hydrobromic acid, sulfuric acid, sulfurous acid, or hydrogen sulfide; or bases, such as potassium hydroxide, sodium hydroxide, magnesium hydroxide, calcium hydroxide, nickel hydroxide, or silver hydroxide. As with the other electrolytes described herein, these materials can be bound together via ionic bonds in their respective undissolved forms, and can be surrounded by molecules of a fluid, such as water, once dissolved. Acids and bases can also form suspensions or slurries with the fluid.

The magnetic field source, which can be located outside of the semi-permeable enclosure, can include a permanent magnet. A permanent magnet can be formed of iron alloy, cobalt alloy, nickel alloy, or another suitable material. The magnetic field source can also include an electromagnet that can induce a magnetic field. The field strength of the electromagnet can be approximated by Ampere's Law as B=μₒηI, where μₒ, is the permeability of the core, η is the number of turns per unit length, I is the current, and B is the magnetic field strength. The magnetic field strength, or B, is directly proportional to the current. The current can be regulated by changing the resistance in the circuit. In some cases, the electric current can flow through an electrode, which may be a graphite electrode. The electrode can further be connected to a wire that may be used to complete the circuit.

Either the permanent magnet or the electromagnet can have the field strength of at least about 1 millitesla (mT), or at least about 2 mT, or at least about 3 mT, or at least about 4 mT, or at least about 5 mT, or at least about 6 mT, or at least about 7 mT, or at least about 8 mT, or at least about 9 mT, or at least about 10 mT, or at least about 15 mT, or at least about 15 mT, or at least about 20 mT, or at least about 25 mT, or at least about 50 mT. In some cases, the magnetic field source can fully or partially surround the fluid current. In some cases, the strength of the magnetic field provided by a magnet can be amplified by plates positioned near the magnet, such as plates formed of steel or iron.

When the magnetic field source fully or partially surrounds the fluid current, a force can be imparted on charged particles within the fluid current. The force F can be determined by the formula F=(qv)(B), where q is the charge of the particle (e.g., +1, +2, +3, −1, −2, −3, and so on), v is the velocity of the fluid current flow (e.g., the velocity of the charged particle), and B is the magnitude of the magnetic field. The force can be orthogonal both to the direction of the magnetic field and to the direction of the fluid current flow, and the direction of the force can vary depending on whether the charge is positive or negative. This force is sometimes called electromotive force (EMF) or a Lorentz force. FIG. 7 schematically illustrates the effect of the Lorenz force on charged particles (where the direction of the magnetic field B is up out of the plane of the page).

The vessel can further include one or more membranes that aid in the concentration the nutrients. The membranes selectively allow passage of charged particles through the membranes. In some cases, a membrane can selectively allow anions to pass through, and block out most or all other species (e.g., cations or uncharged species). Such a membrane may be called an anion-selective membrane. In other cases, a membrane can selectively allow cations to pass through, and block out most or all other species (e.g., anions or uncharged species). Such a membrane can be called a cation-selective membrane. In some cases, anion-selective membranes can be positively charged, and cation-selective membranes can be negatively charged. Anions and cations can be driven toward the anion-selective membrane and the cation-selective membrane, respectively, by the Lorenz force acting on the anions and the cations. In some cases, anions that have passed through an anion-selective membrane and cations that have passed through the cation-selective membrane flow through two separate channels that are then joined in a single channel where the cations and anions are combined. In such cases, the concentration of anions and cations in the single channel can be higher than in the fluid current flow before it experienced the influence of a magnetic field. The anions and cations can be further directed into the semi-permeable enclosure, for example via a passageway or a pipe. In some cases, each of the channels can have one or more electrodes, which can be used to provide a current for a magnetic field. In some embodiments, each channel can have two oppositely charged electrodes. In some cases, the electrodes can be parallel to one another.

In contrast to the charged particles, uncharged particles (e.g., molecules of the fluid solvent, such as water, or oil molecules, such as hydrocarbon molecules), may not experience a Lorentz force and may not deviate from the direction of the current flow as may charged particles schematically depicted in FIG. 7. Such particles may not pass through the cation or anion-selective membranes, and can become directed to a different channel or channels than the charged particles. Such particles can further be directed away from the semi-permeable enclosure, for example via pump or a pipe. In some cases, such particles can be directed back into the aquatic environment as part of a discharged fluid. The discharged fluid can have a lower anion and cation concentration that in a fluid current flow before it experienced the influence of a magnetic field. In some embodiments, a sensor or a probe can be located downstream of a location where a fluid current passes the influence of a magnetic field, so that changes in electrolyte, including ion, concentrations can be measured. The sensor or probe can also measure the rate of increase or decrease of electrolyte concentration over time. For example, a probe can be inserted into the channel or channels expected to have an increased concentration of electrolytes, or into the channel or channels expected to have a depleted concentration of electrolytes. Some probes can be equipped to measure concentrations of a specific ion, such as nitrate or phosphate.

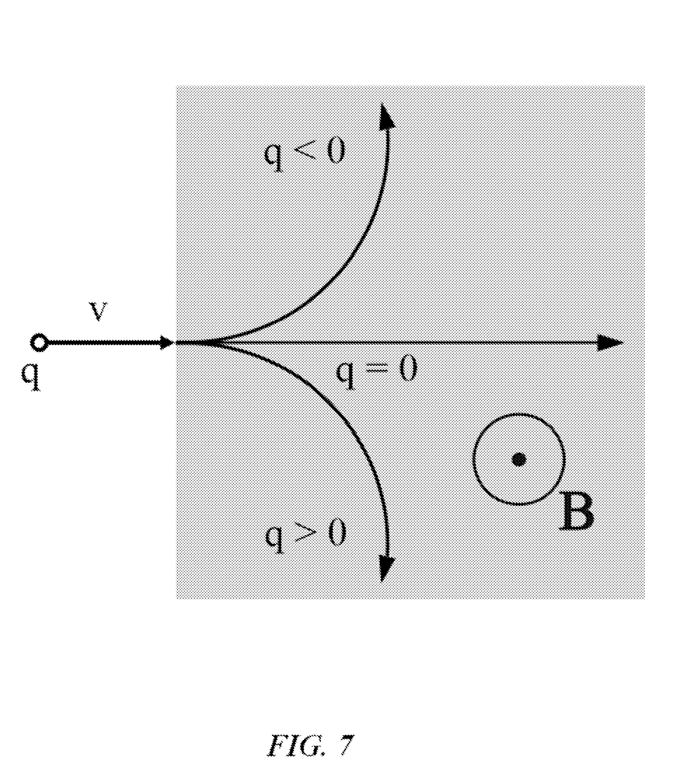

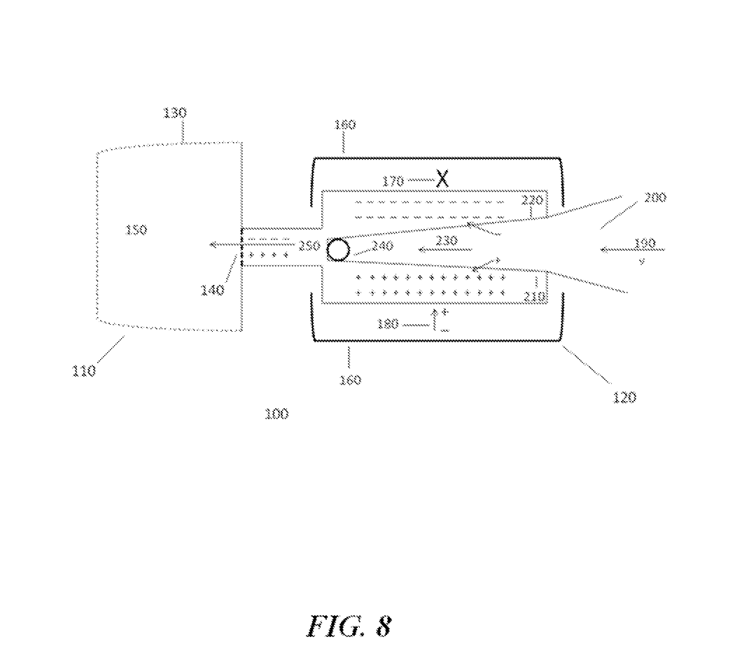

FIG. 8 schematically illustrates a vessel that includes a bioharvester and a magnetic field source, in accordance with an embodiment of the invention. The vessel 100 includes a bioharvester module 110, and magneto-concentrator module 120. The bioharvester module 110 can be any of the vessels described herein, such as any of the vessels of FIGS. 4-6. The bioharvester module 110 includes a semi-permeable membrane 130, a gate 140, which may be a sliding gate, and an enclosure 150, where microorganisms such as algae may grow. Magneto-concentrator module 120 may include a magnetic field source 160, which may be a permanent magnet or an electromagnet, and which may partially or fully surround other components of the magneto-concentrator module 120. Magnetic field source 120 may generate a magnetic field 170, depicted herein by an “X” which indicates that the direction of magnetic field 170 is into the plane of the page. Magnetic field 170 generates a Lorenz force 180 on charged particles that enter the magneto-concentrator module 120 via a fluid current 190, which may have velocity “v.” A channel 200 may increase the velocity v of the fluid current. The channel 200 can be a narrowing channel, having a width (as measured along an axis orthogonal to the direction of flow) that decreases along the direction of flow. Once inside the magneto-concentrator module 120, charged particles in the fluid current (positively charged particles are indicated by “+” and negatively charged particles are indicated by “−”) experience Lorenz force 180, which drives negatively charged and positively charged particles in opposite directions. Positively charged particles may pass through a cation-permeable membrane 210 and negatively charged particles may pass through an anion-permeable membrane 220, as indicated by the arrows. A fluid with reduced anion and cation (e.g., electrolyte) concentration (relative to electrolyte concentration in the fluid current 190) continues to flow in the diluent channel 230 and is removed from vessel 100 via a discharge pipe 240 (circle indicates flow direction of the discharged fluid is out of the plane of the page). Cations and anions that have passed the cation- and anion-permeable membranes, respectively, enter electrolyte channel 250, which carries the fluid with an increased electrolyte concentration relative to electrolyte concentration in the fluid current 190. As electrolytes are being collected from electrolyte channel 250 into bioharvester module 110, gate 140 may be open. After collection is complete, gate 140 may be closed so that bioharvester module 110 and magneto-concentrator module 120 may be isolated from one another. Some electrolytes thus collected may include nutrients, and they may aid in the growth of microorganisms, such as algae, within enclosure 150.

In another aspect, a system comprises a first module comprising a buoyant top, a buoyancy-controlled base, and a semi-permeable enclosure connecting the buoyant top to the buoyancy-controlled base. The first module can be adapted to retain one or more microorganisms upon the flow of a fluid stream through the first module. The system further comprises a second comprising a magnetic field source that is configured to provide a magnetic field into the second module, such that the second module is adapted to concentrate ionic species upon the flow of the ionic species or a fluid having the ionic species through the second module along a direction generally orthogonal to the magnetic field. The magnetic field source can be as described elsewhere herein. The second module can be adjacent to the first module. In some cases, the second module is not adjacent the first module. For example, the second module can be disposed remotely with respect to the first module, such as at a different depth and/or lateral location than the first module. Ionic species concentrated in the second module can be directed to the first module with the aid of a pumping system and one or more channels bringing the first module in fluid communication with the second module.

In some embodiments, the first module and the second module can be separated by a distance. The distance can be about 1 cm, or about 10 cm, or about 1 m, or about 10 m, or about 100 m, or 500 m about 1 km, or about 2 km, or about 3 km, or about 4 km, or about 5 km, or longer. In some cases, the first module can be positioned at the same depth, e.g., the same depth in the aquatic environment, as the second module. In other cases, the first module can be positioned at a different depth than the second module. In an example, the second module can be positioned deeper than the first module. In some cases, the first module can be positioned closer the fluid surface, while the second module can be positioned closer to the floor of the fluid, such as, for example, ocean floor. In some cases, the second module can be configured in such a way as to withstand fluid pressure. In some embodiments, the second module can be positioned in an area of the aquatic environment where fluid current velocity is high relative to other areas of the aquatic environment. Fluid current velocity can be measured by techniques known in the art, such as described in, for example, U.S. Pat. No. 6,820,008 to van Smirren et al., which is entirely incorporated herein by reference. In some embodiments, the second module can be positioned in an area of the aquatic environment where electrolyte concentration is high relative to other areas of the aquatic environment. Electrolyte concentration can be measured by sensors known in the art, such as described in, for example, U.S. Patent Publication. No. 2008/0302660 to Kahn et al., which is entirely incorporated herein by reference. Such sensors can be electrically coupled to a control system that is adapted to regulate the buoyancy of one or both of the modules.

The buoyancy of the first and second modules can be regulated with the aid of a device that regulates depth, such as, for example, a gas tank that operates under Archimedes' principle. The control system can be coupled to sensors that measure ionic concentration to regulate the depth of the second module to aid in optimizing ion capture. For example, the depth of the second module can be selected such that the concentration of ions is increased or maximized in relation to another depth.

Concentrated ions from the second module may be directed to the first module either manually (e.g., manually removing the ions from the second module), or with the aid of a fluid flow system that directs the concentrated ions to the first module. The fluid flow system can comprise a pipe or channel that brings the first module in fluid communication with the second module. The fluid flow system can include a pump for facilitating fluid flow.

The first module can be a bioharvester module, such as that depicted in FIG. 8 (e.g., bioharvester 110). Microorganisms such as algae may be grown in the first module, a semi-permeable membrane may enable the first module to selectively retain microorganisms from a fluid stream, such as a seawater or river current.

The second module may include a magneto-concentrator, such as that depicted in FIG. 8 (e.g., magneto-concentrator 120). As depicted in FIG. 8, the direction of the fluid current, including that of ionic species (e.g., electrolytes), is orthogonal to the direction of the magnetic field generated by a magnetic field source. Ionic species may be concentrated with the aid of cation- and/or anion-selective membranes, in the manner shown in FIG. 8 (e.g., membranes 210 and 220) and described herein. The magnet may be a permanent magnet or an electromagnet, and have a field strength of at least about 1 millitesla (mT), or at least about 2 mT, or at least about 3 mT, or at least about 4 mT, or at least about 5 mT, or at least about 6 mT, or at least about 7 mT, or at least about 8 mT, or at least about 9 mT, or at least about 10 mT, or at least about 15 mT, or at least about 15 mT, or at least about 20 mT, or at least about 25 mT, or at least about 50 mT. The magnet may partially or fully surround the second module. The magnet may be in the form of a coil, as described in PCT/US2012/041766, which is entirely incorporated herein by reference.

In some cases, the second module may be attached to the first module. In other cases, the second module may be coupled to the first module via a pipe or a channel. The pipe or channel may be configured to direct fluid flow from the second module to the first module. In some cases, the pipe or channel may direct a fluid including concentrated ionic species from the second module to the first module, as in the manner depicted in FIG. 8 (e.g., from magneto-concentrator 120 to bioharvester 110). Concentrated ionic species may also be actively transported from the second module to the first module via a third module, which may be a device such as a pump. The second module may also include a pipe, channel, or pump for discharging a fluid with reduced concentration of ionic species into the aquatic environment.

In some cases, the semi-permeable membrane of the first module is collapsible or compressible. In some cases, the buoyancy-controlled base of the first module is movable with respect to the buoyant top, as described herein.

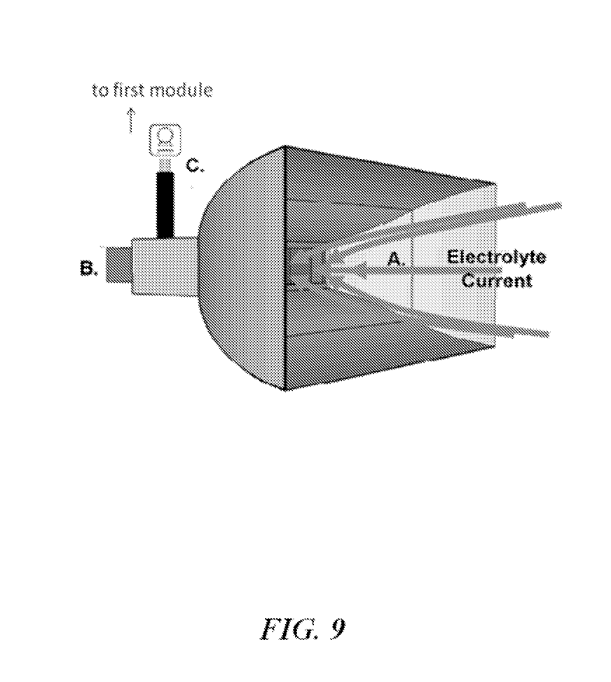

FIG. 9 schematically illustrates an example of a second module. A current of fluid, including electrolytes, flows into the second module through a channel (A), which may increase the velocity of the current and the electrolytes within the current. After the magnetic field source operates on the electrolyte current to concentrate electrolytes in the manner described herein, the fluid with a reduced concentration of electrolytes flows out of the second module via a discharge pipe (B). The concentrated ionic species are collected into a pipe (C) and driven with the aid of a pump into the first module.

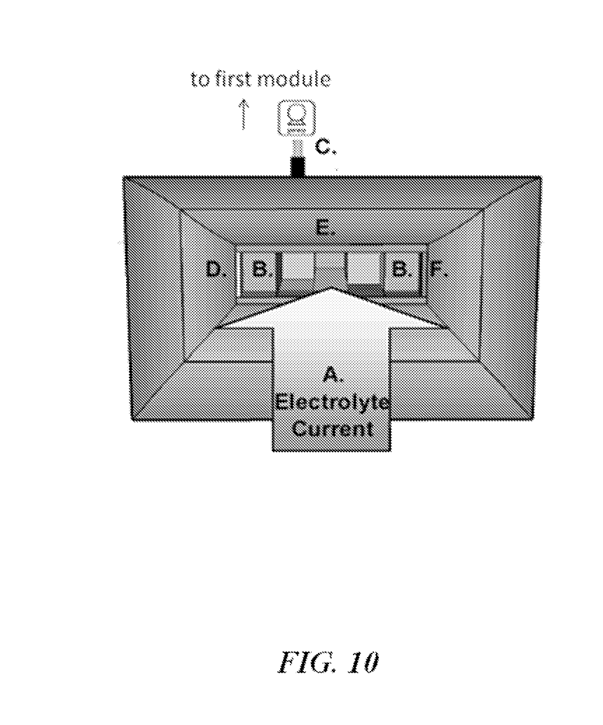

FIG. 10 provides another schematic view of a second module. A current of fluid, including electrolytes, flows into the second module through a channel (A), which may increase the velocity of the current and the electrolytes within the current. A magnetic field source (E) may surround the channel. The magnetic field creates a Lorentz force such that a dipole is effectively formed across the second module, with a “cathode” region (D) to which cations are attracted, and an “anode” region (F) to which anions are attracted. The concentrated ionic species are collected with into a pipe (C) and driven with the aid of a pump into the first module. The fluid with a reduced concentration of electrolytes flows out of the second module via a discharge pipe (B).

FIG. 11 provides yet another schematic view of a second module. A current of fluid, including electrolytes, flows into the second module through a channel (A), which may increase the velocity of the current and the electrolytes within it. A magnetic field source (E) may surround the channel. The concentrated ionic species are collected with into a pipe (C) and driven with the aid of a pump into the first module. The fluid with a reduced concentration of electrolytes flows out of the second module via a discharge pipe (B).

Methods for Collecting and/or Generating Biomass

Another aspect provides methods for collecting and/or generating biomass. A method for collecting and/or harvesting biomass can include directing a fluid stream from a first module to a second module along a first fluid flow path. The fluid stream is directed through a movable gate of the first module. The movable gate is adapted to regulate fluid flow (i) along the first fluid flow path and (ii) along a second fluid flow path leading from the second module to the first module. Next, at least a portion of the fluid is directed from the second module to the first module along the second fluid flow path. Next, at least a portion of the fluid is directed from the second module to a third module. The third module includes one or more surfaces for retaining one or more microorganisms upon the flow of the at least the portion of the fluid through the third module.

In some cases, a method for collecting and/or generating biomass includes providing a microorganism from an aquatic environment into a vessel configured to retain the microorganism, with the vessel comprising at least one semi-permeable membrane that permits the unidirectional flow of the microorganism through the membrane, concentrating one or more nutrients from the aquatic environment with the aid of a magnetic field applied to a fluid stream flowing from the aquatic environment into the vessel, and providing the one or more concentrated nutrients into the vessel. The semi-permeable membrane can allow fluid from the aquatic environment to pass freely while impeding diffusion of the microorganism out of the vessel.

The nutrients can be water-soluble. The nutrients can include electrolytes, such as salts, acids, and basis, as described herein. In water or another fluid, such as a fluid providing a fluid current, electrolytes can be dissociated into cations and anions. In some cases, the electrolytes can function as nutrients, aiding, for example, in the growth of microorganisms such as algae. Nutrient cations in fluid streams that can be concentrated with the aid of a magnetic field source include sodium (Na⁺), potassium (K⁺), magnesium (Mg²⁺), aluminum (Al³⁺), ammonium (NH₄⁺) and other cations as described herein. Nutrient anions in fluid streams that can be concentrated with the aid of a magnetic field include nitrate (NO₃⁻), phosphate (PO₄³⁻), chloride (Cl⁻), acetate (H₃C(O)O⁻), carbonate (CO₃⁻) and other anions as described herein.

In some cases, the magnetic field applied to a fluid stream is provided by a permanent magnet. In other cases, the magnetic field applied to a fluid stream is provided by an electromagnet. The permanent magnet or electromagnet can be in the form of a coil.

An aquatic environment can include a natural aquatic environment such as river water, seawater, ocean water, or lake water, or a man-made aquatic environment such as a pool, algae farm, or tank. In some cases, the aquatic environment can provide its own (e.g., natural) electrolytes and/or nutrients. In other cases, the electrolytes and/or nutrients can be provided from an external source, which can include recycled nutrients. The nutrients from the aquatic environment can be concentrated in the vessel with the aid of a magnetic field and with the further aid of one or more ion-selective membranes, which can include a cation-selective membrane and an anion-selective membrane. The nutrients can be driven into the vessel with the aid of a pipe and/or a pump, and retained in the vessel with the aid of the vessel's semi-permeable membrane. After the concentration of one or more nutrients in the vessel, a fluid, such as water, with a reduced concentration of nutrients can be released back in to the aquatic environment.

Ion-selective membranes can include polymeric species, such as materials used in electrodialysis applications. For example, an anion-selective membrane can include metalloporphyrins or metallo-crown ethers bound in a polymeric array, while a cation-selective membrane can include carbonate, phosphate, or acetate groups bound in a polymeric array. The polymer backbone of anion- or cation-selective membranes can include a polyethylene, polypropylene, polystyrene, or polystyrene-pyridine co-polymer.

In some cases, the one or more ion-selective membrane(s) can operate chemoselectively. The membranes can permit nutrients such as nitrates and phosphates to pass through into the vessel, but may not permit electrolytes that may be less nutritively valuable, such as chlorides, to pass through into the vessel. In some embodiments, the membranes can include one or more zeolites or other functionalities or surface-active agents that are configured to trap, for example, chloride ions preferentially over other ions such as phosphates or nitrates. Such agents may include sodalite, zeolite A, zeolite XY, and zeolite Y. In some cases, chloride ions can be trapped preferentially over nitrates or phosphates in part because of the size difference between the smaller chloride ion relative to the larger phosphate or nitrate ions, such that the latter (e.g., phosphate, nitrate) might not fit into the zeolite structure. In such cases, the membrane, such as an anion-selective membrane, can still function to separate anions from cations because anions will be attracted to the positive charge on the zeolite, but the larger anions (e.g., phosphate, nitrate), will not be trapped and pass through to the vessel, while the smaller anions (e.g., chloride) may be trapped in the zeolites. In some cases, zeolitic structures can be incorporated into polymeric backbone of the anion- or cation-selective membranes.

In some embodiments, ions pass through the ion-selective membranes on the basis of their charge. In an example, magnetic field strength can be adjusted (e.g., reduced or increased) in such a way that the Lorenz force is sufficient to enable only trivalent ions (e.g., phosphate) to pass through an ion-selective membrane, while the Lorenz force on monovalent ions (e.g., chloride) may not be sufficiently strong to enable a chloride ion to pass through an ion-selective membrane. In other cases, the magnetic field strength can be adjusted such that certain ions are selectively separated in relation to other ions. For example, trivalent ions can be selectively separated in relation to monovalent and/or divalent ions by appropriately selecting the magnetic field strength. In some cases, magnetic field can be adjusted throughout the period that ions are collected to permit selective concentration of monovalent, divalent, trivalent, or tetravalent cations at desired times. In some cases, higher-valent ions, such as phosphates, can have higher nutritive value than lower-valent ions, such as chlorides. Selective ion separation in some cases can be facilitated with the aid of ion permeable membranes that are selected to permit only certain ions to pass through.

A microorganism can include algae, microalgae, plankton, diatoms, phytoplankton, zooplankton, or other species as described herein. The microorganisms naturally occur in the aquatic environment, or can be provided from an external source into the aquatic environment. The microorganisms can flow into the vessel from the aquatic environment in a fluid stream, or can be placed into the vessel by a human operator.

An aspect of the disclosure provides a method for recycling one or more agricultural fertilizers. The method includes providing an agricultural runoff, filtering the agricultural runoff to form a first fluid, concentrating one or more fertilizers from the first fluid with the aid of a magnetic field applied to the first fluid, thereby forming a second fluid comprising one or more concentrated fertilizers. The second fluid comprising one or more concentrated fertilizers can then be collected.

Electrolytes such as fertilizers can also be removed from fluids using methods and modules (e.g., magnetic concentrator modules) described herein. Electrolytes can be concentrated and/or removed from fluids using methods and modules (e.g., magnetic concentrator modules) described herein. In some cases, electrolyte concentration in a fluid containing one or more electrolytes, such as water containing fertilizers from agricultural run-off, can be reduced with the aid of a magnetic concentrator. Fertilizers can include nitrates and phosphates. In some cases, the fluid can be filtered before coming under the influence of a magnetic field source. The fluid can be filtered through, for example, clarifier plates.

A magnetic concentrator utilizing an applied magnetic field can be used to separate a fluid into a first stream comprising a high electrolyte concentration and a second stream comprising a low electrolyte concentration. In an example, a unit such as magnetic concentrator 120 depicted in FIG. 8 may be used, with except that the concentrated electrolyte stream 250 may be recovered not in a bioharvester, but in a different kind of unit, such as for example, a tank for collecting fluid, and that the depleted electrolyte stream 230 may be recovered in a different tank rather than discharged into the aquatic environment. In some cases, the fluid can be filtered to remove some solids before reaching the magnetic concentrator.

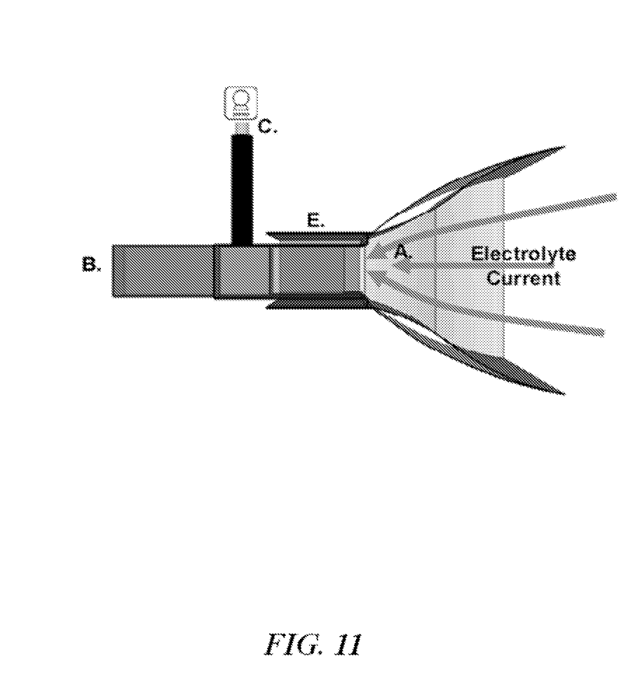

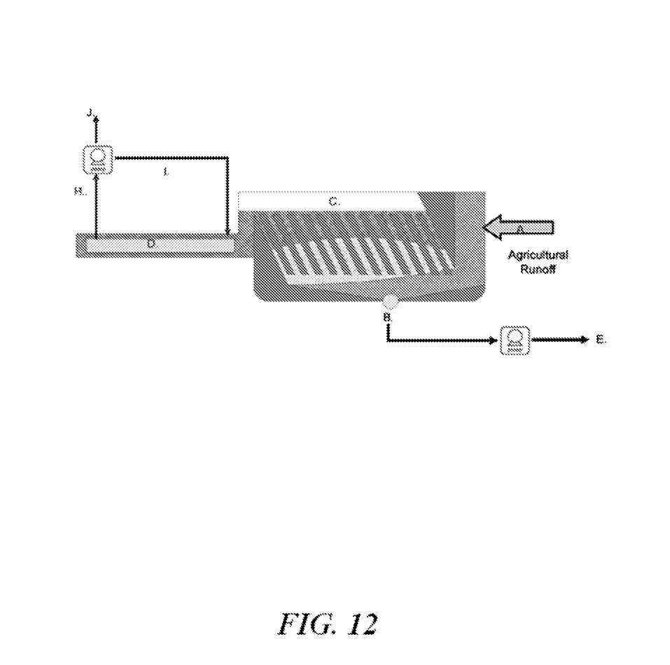

FIG. 12 schematically illustrates a fertilizer recycler, in accordance with an embodiment of the invention. Agricultural runoff (A) enters a chamber comprising clarifier plates (C) and solids drainage point (B). Clarifier plates filter away some of the solids in the agricultural runoff, which enters the drainage point. The resulting solids sludge (E) is removed from the chamber via a pump. The filtered fluid enters fertilizer concentrator chamber (D), which can then come under an influence of a magnetic field. Upon operation of a magnetic field, concentrated electrolyte stream (H) is recovered via a pump to make concentrated fertilizer (J), which may be reused. The concentrated electrolyte may be partially recycled in the system via loop (I). The fertilizer of FIG. 12 can be used in any of the vessels described herein, such as the inclined plate module 404 of FIGS. 4A and 4B of the settler module of FIGS. 5A-5C.

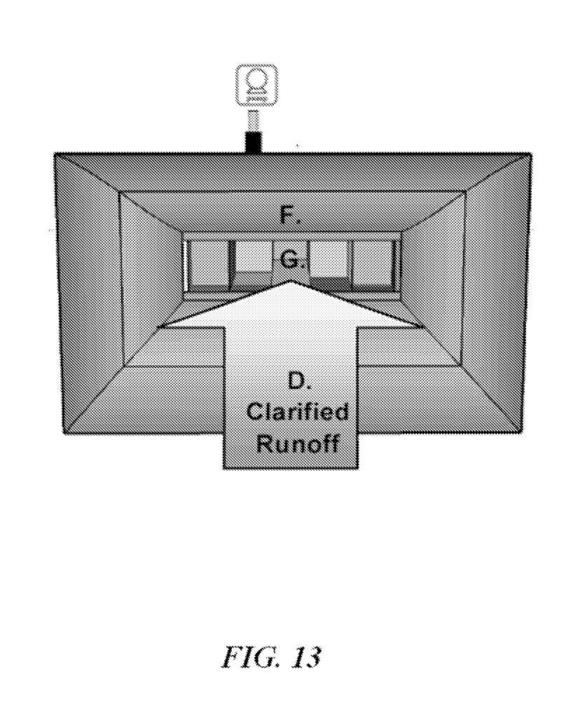

FIG. 13 provides another view of a fertilizer recycler. A clarified fluid enters a fertilizer concentrator chamber (D), and then comes under an influence of a magnetic field source (F), creating a diluted stream (G) and a concentrated stream, such as that shown as (H) in FIG. 12. Diluted stream (G) may be discharged into the aquatic environment or reused (as, for example, potable water).

### Example 1

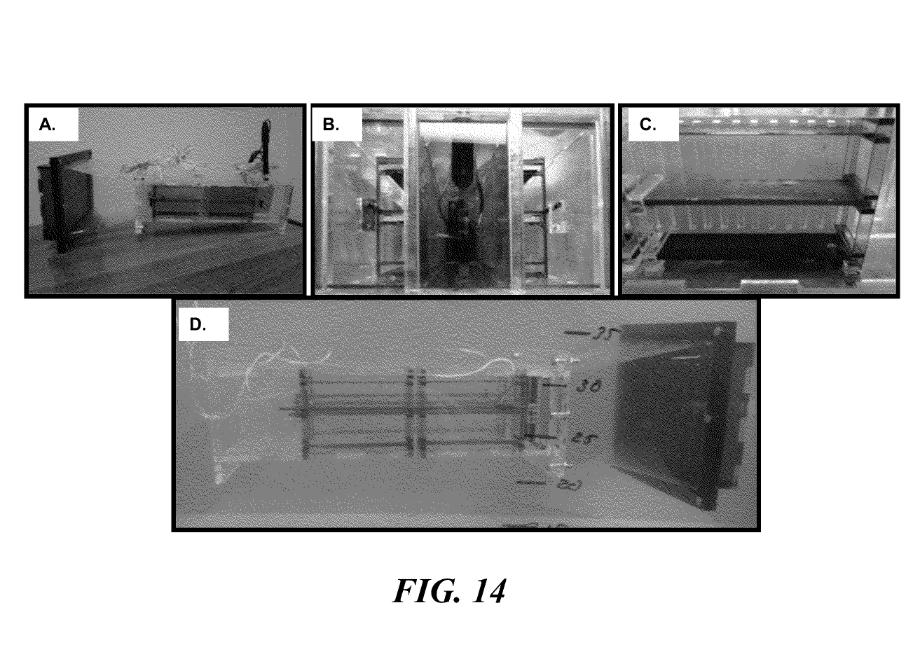

FIGS. 14A-14D show an example of a MHFC. FIG. 14A shows an assembled MHFC illustrating the black fluid channeling backing plate and fluid funnel (left), an electrodialysis stack (right), insulated copper leads from the sacrificial graphite electrodes, and the black nitrate ion selective electrode protruding from a concentration chamber in the electrodialysis stack. FIG. 14B shows a three channel electrodialysis stack with a dilution channel on either side of the center concentration channel. The dilution channels contain a neodymium rare earth magnet array and a graphite electrode. The concentration channel/chamber contains an electrode on either side of an insulated partition. The nitrate ion selective electrode is inserted in the concentration chamber downstream of the channel's electrodes in order to continuously measure the change in nitrate concentration in the chamber. FIG. 14C shows 1 of 6 of the neodymumium magnet arrays in the in the dilution channels and slits between the dilution and concentration chamber. FIG. 14D shows the MHFC suspended in the 75 gallon closed water test bed.

### Example 2

Thirty microalgal strains were screened for their biomass productivity and lipid content. Four strains (two marine and two freshwater), selected because they were robust, highly productive and had a relatively high lipid content, were cultivated under nitrogen deprivation in 0.6-L bubbled tubes. Only the two marine microalgae accumulated lipid under such conditions. One of them, the eustigmatophyte Nannochloropsis sp. F&M-M24, which attained 60% lipid content after nitrogen starvation, was grown in a 20-L Flat Alveolar Panel photobioreactor to study the influence of irradiance and nutrient (nitrogen or phosphorus) deprivation on fatty acid accumulation. Fatty acid content increased with high irradiances (up to 32.5% of dry biomass) and following both nitrogen and phosphorus deprivation (up to about 50%). To evaluate its lipid production potential under natural sunlight, the strain was grown outdoors in 110-L Green Wall Panel photobioreactors under nutrient sufficient and deficient conditions. Lipid productivity increased from 117 mg/L/day in nutrient sufficient media (with an average biomass productivity of 0.36 g/L/day and 32% lipid content) to 204 mg/L/day (with an average biomass productivity of 0.30 g/L/day and more than 60% final lipid content) in nitrogen deprived media. In a two-phase cultivation process (a nutrient sufficient phase to produce the inoculum followed by a nitrogen deprived phase to boost lipid synthesis) the oil production potential could be projected to be more than 90 kg per hectare per day. There was an increase of both lipid content and areal lipid productivity through nutrient deprivation in an outdoor algal culture. The experiments show that marine eustigmatophyte can produce 20 tons of lipid per hectare per year in the Mediterranean climate and more than 30 tons of lipid per hectare per year in sunny tropical areas.

### Example 3

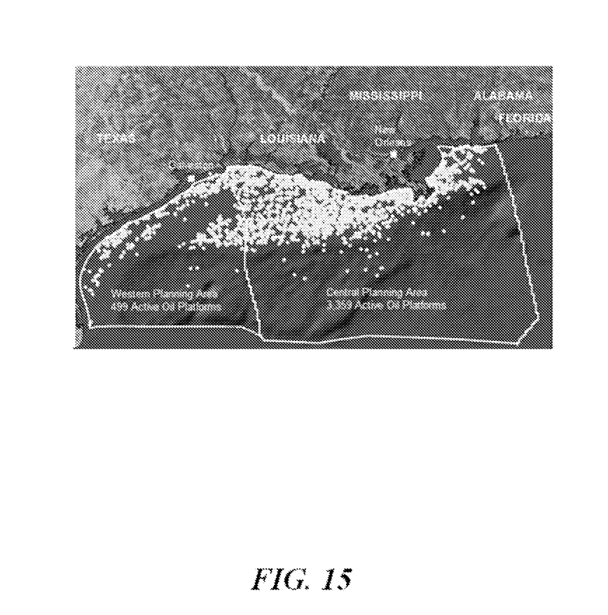

Iron fertilization vessels and concentrators are used around oil drilling platforms off the United States Gulf of Mexico coastlines. FIG. 15 shows the locations of oil rigs off of the United States Gulf coasts that are within 50 km from the shore. The waters near the Mississippi river region offer surface waters that are rich in nitrates and phosphates that have been drained from farms and cities that line the river and its tributaries in the central United States. Once the fertilizer sequestration and concentration mechanism is developed, the fertilization vessels can be used in nitrate/phosphate deficient surface waters. FIG. 3B shows iron fertilization vessels around a drilling platform. The platform offers a location to harvest, pre-process and separate the microalgae into algae oil and protein precipitate. A tanker retrofitted with the tools necessary for converting the microalgae to renewable fuels at sea is also a viable option.

### Example 4

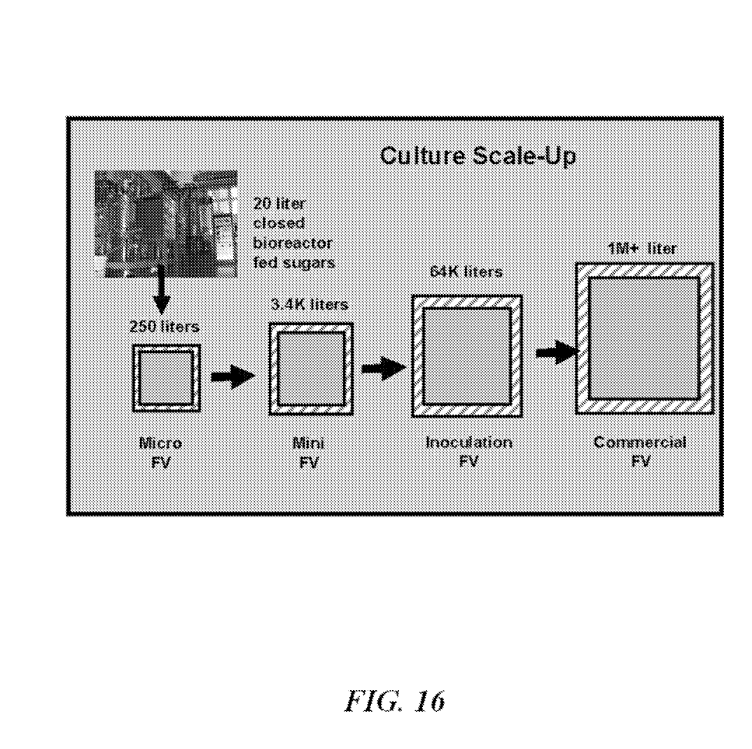

FIG. 16 shows an approach for scaling up a bioharvester, which may be any of the vessels of the disclosure. Initially, a seed culture is grown in a completely sterile 20 L bioreactor. The 20 L seed culture is used to inoculate the 250 L micro fertilization vessel. Once the culture matures in the micro fertilization vessel, its contents are transferred to the 3400 L (3.4K liters) mini fertilization vessel. This process of growth and transferring to a larger vessel is repeated until the commercial scale of over one million liter fertilization vessel is inoculated. The volume of the fertilization vessel can be manipulated by inserting or removing extension modules, such as those in FIGS. 4-6. The experiments in this example focus on the growth of dense culture of Nannochloropsis biomass and the ability to scale the cultures.

### Example 5

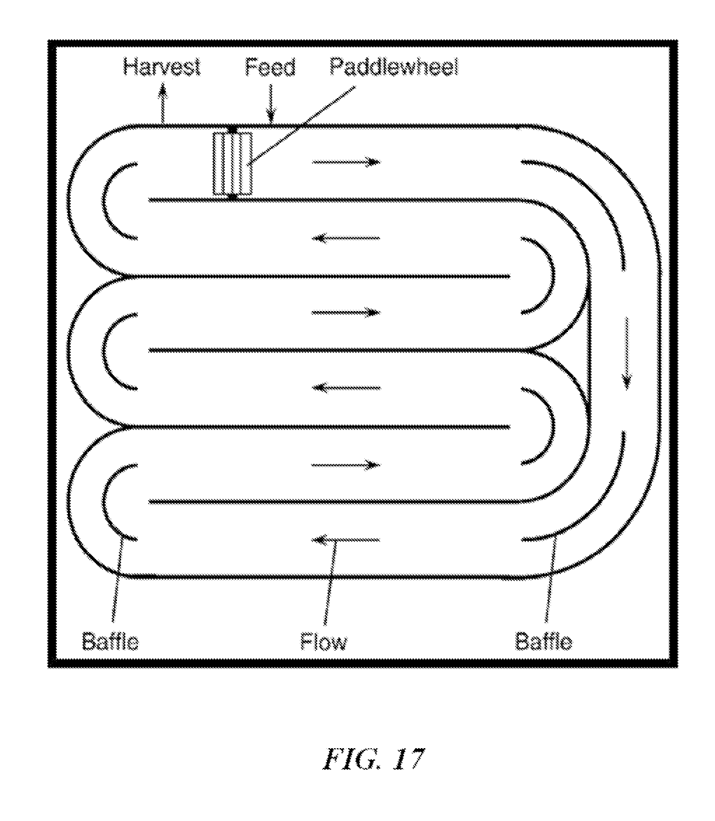

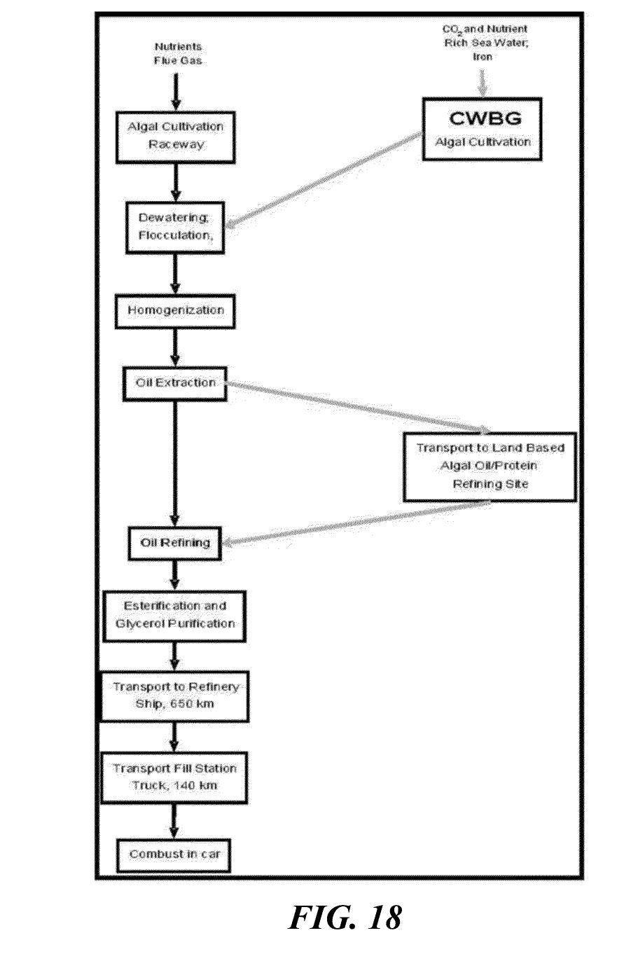

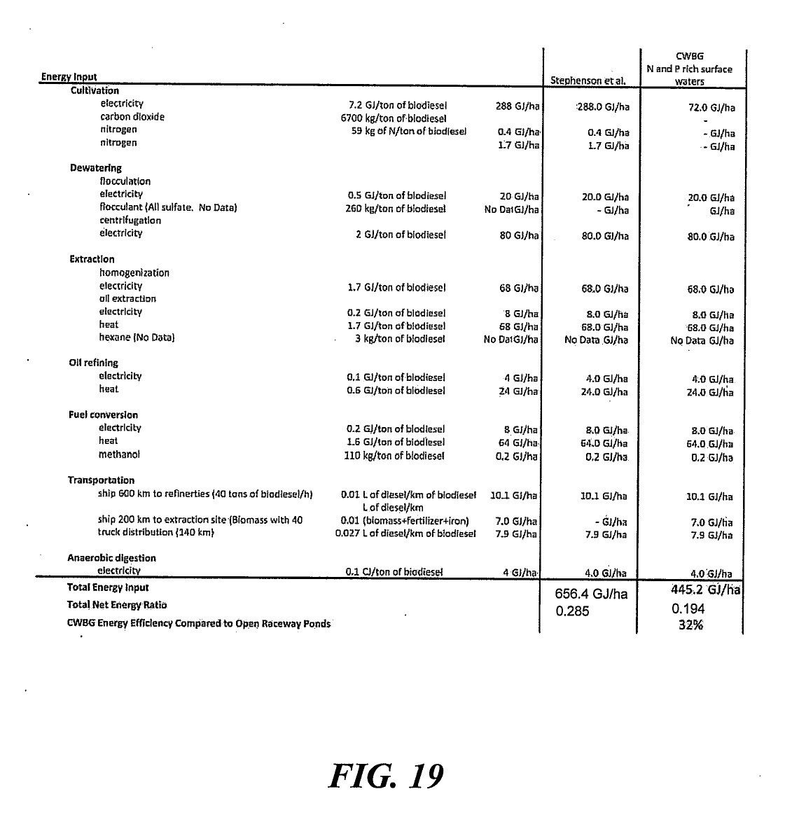

FIG. 17 shows an aerial view of a typical open raceway pond (ORP) system. Seventy-five percent of the ORP energy requirements during the cultivation phase are used to circulate the culture by the paddlewheel. In comparison, fertilization vessels of the disclosure can use ocean currents to circulate a culture, thus reducing the overall cultivation energy requirements. FIG. 18 is a process flow diagram for cultivating biomass using vessels of the disclosure (CWBG) as compared to a typical ORP system. Vessels of the disclosure can cultivate algae using dissolved bicarbonates present in seawater without the use of an ORP. FIG. 19 shows a table with energy savings using vessels of the disclosure (CWBG) as compared to ORP systems.

### Example 6

A Nannochloropsis oculata is cultured in a vessel similar to the vessel 500 of FIG. 5 that is suspended in a 75 gallon closed water aquarium. The temperature of the aquarium is maintained at 25° C. under continuous illumination. The aquarium is aerated with 2,800 μl⁻¹ CO₂ at 200 ml min⁻¹, and grown to its late growth phase. Instant Ocean is added to achieve a salinity of about 35 ppt. A liter of artificial seawater enriched with 4 mM Nitrate and 0.1 nM Phosphate is fed to the vessel based on the feedback from a nitrate ion selective electrode placed immediately before an exit orifice of the vessel. As long as the nitrate exit levels are less than 40 μM Nitrate, fertilizer is fed directly to the suspended fertilization vessel and controlled by a custom Arduino module. The artificial seawater is pumped directly into the fertilization vessel via a custom plate attached to the upstream module.

Cell density is measured turbidimetrically at 750 nm and converted from an appropriate calibration curve to ash free dry mass (AFDW). For the determination of water and ash content, 6-12 mg of freeze-dried biomass (W0) are placed on a pre-ashed aluminum foil weigh boat and weighted (W2) and then dried at 100° C. for at least 12 hours. After weigh-in (W2), samples are heated at 540° C. to combust all organic carbon, with the weight of the foil weigh boat and inorganic residues (W3). Water and ash content (w/w) are then determined by dividing the respective weight difference (W1−W2)/W0 and (W2−W3)/WO. All cultures are initiated with an OD750 of about 0.1 OD750 was monitored every 24 hrs until a stationary phase is reached.

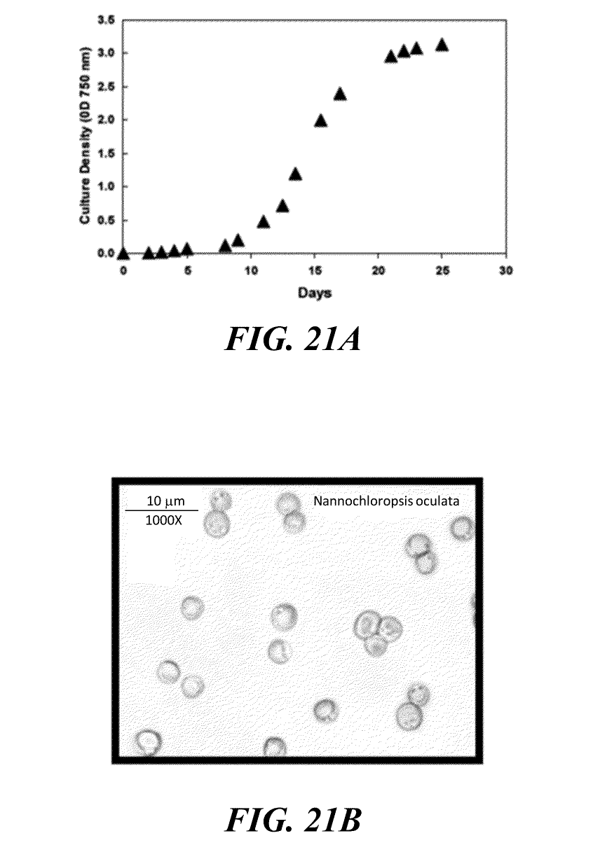

The nannochloropsis oculata growth profile is shown in FIG. 21A. Culture density (OD, 750 nm) is along the y-axis, and time (days) is along the x-axis. FIG. 21B is a light microscope image of the nannochloropsis oculata from a sample taken from the vessel during the exponential phase (between about 12-17 days) of the growth illustrated in FIG. 21A. These results show that algae can be contained and cultivated in a cultivation section of the vessel. Cells that escape the cultivation section of the fertilization vessel flocculate, settle and concentrate in the settler module. The maximum optical density during the stationary phase is an order of magnitude lower than the literature values.

Systems and methods of the disclosure may be combined with or modified by other systems and methods, such as, for example, those described in Kalra, A. and W. S. LLP. (2006), “BiodieselTax Credits”; Walford, L. A., “Living Resources of the Sea: Opportunities for Research and Expansion,” New York, Ronald Press (1958); Rodolfi et al., “Microalgae for Oil: Strain Selection, Induction of Lipid Synthesis and Outdoor Mass Cultivation in a Low-Cost Photobioreactor,” Biotechnology and Bioengineering, Vol. 102, No. 1, Jan. 1, 2009 (Wiley Periodicals, Inc.); Loscher, B. M., “Relationships among Ni, Cu, Zn, and major nutrients in the Southern Ocean,” Marine Chemistry 67: 67-102 (1999); Patent Cooperation Treaty (PCT) Patent Publication No. WO/2010/141559 (“SYSTEMS AND METHODS FOR CULTIVATING, HARVESTING AND PROCESSING BIOMASS”); and PCT Application No. PCT/US2012/041766, each of which is entirely incorporated herein by reference.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents. It is intended that the following claims define the scope of the invention and that methods and structures within the scope of these claims and their equivalents be covered thereby.

What is claimed is:

1. A collector of biomass, comprising:
(a) a vessel, comprising:
(i) one or more surfaces for collecting a biomass from a fluid directed through said vessel; and
(ii) one or more internal impellers in fluid communication with said one or more surfaces through a fluid flow path, wherein said one or more internal impellers facilitate flow of said fluid through said fluid flow path; and
(b) an external impeller coupled to said one or more internal impellers, wherein said external impeller is disposed external to said vessel and adapted to provide rotational energy to said internal impeller upon fluid flow through or adjacent to said external impeller.

2. The collector of claim 1, wherein said one or more surfaces are part of one or more plates.

3. The collector of claim 1, wherein said vessel comprises a magnet or electromagnet that is at least partially enclosed by an exterior wall of said vessel.

4. The collector of claim 1, further comprising a self-orienting mechanism capable of orienting the direction of the vessel with respect to the direction of a current flow when the vessel is positioned in a current.

5. The collector of claim 1, wherein said fluid flow path is a circulatory fluid flow path.

6. The collector of claim 1, wherein said vessel comprises a first module and a second module, wherein said second module is downstream of said first module along said fluid flow path extending from said first module to said second module, and wherein said first module includes said one or more internal impellers.

7. The collector of claim 6, further comprising a gate that at least partially isolates said fluid flow path.

8. The collector of claim 7, wherein said gate is a movable gate.

9. The collector of claim 6, further comprising a membrane/sieve in said first module, wherein said membrane/sieve is included in said fluid flow path.

10. The collector of claim 1, wherein said vessel comprises a plurality of modules, and wherein said one or more surface and said one or more internal impellers are disposed in separate modules.

11. A system for collecting and/or harvesting biomass, comprising:
a. a first module adapted to accept a fluid stream, wherein said first module includes a gate that is adapted to regulate the flow of said fluid stream;
b. a second module downstream of said first module, wherein said second module accepts a fluid from said first module and directs at least a portion of said fluid to said first module; and
c. a third module downstream of said second module, wherein said third module includes one or more surfaces for retaining one or more microorganisms upon the flow of at least a portion of said fluid stream through said third module.

12. The system of claim 11, wherein said one or more surfaces are part of one or more plates.

13. The system of claim 11, wherein said first module and second module are separable from one another.

14. The system of claim 11, wherein said second module and third module are separable from one another.

15. The system of claim 11, wherein said first module includes one or more impellers for facilitating fluid flow through said first and second modules.

16. The system of claim 15, further comprising an external impeller that is external to said first, second and third modules, wherein said external impeller is coupled to said internal impeller and imparts rotational motion to said one or more internal impellers upon fluid flow through or adjacent to said external impeller.

17. The system of claim 11, further comprising a fourth module between said first and second modules, wherein said fourth module extends a length of a fluid flow path from said first module to said second module.

18. The system of claim 17, wherein said fourth module includes an optical window for permitting electromagnetic radiation for coming in contact with at least a portion of said fluid stream.

19. The system of claim 11, further comprising a nutrient concentrator upstream of said first module, wherein said nutrient concentrator is adapted to concentrate one or more nutrients in said fluid stream prior to said fluid stream entering said first module.

20. The system of claim 19, wherein said nutrient concentrator includes a magnetic field source that is adapted to induce a magnetic force that concentrates said one or more nutrients.

21. A method for collecting and/or harvesting biomass, comprising:
a. directing a fluid stream from a first module to a second module along a first fluid flow path leading from said first module to said second module, wherein said fluid stream is directed through a movable gate of said first module, and wherein said movable gate is adapted to regulate fluid flow (i) along said first fluid flow path and (ii) along a second fluid flow path leading from said second module to said first module;
b. directing at least a portion of said fluid from said second module to said first module along said second fluid flow path; and
c. directing at least a portion of said fluid from said second module to a third module, wherein said third module includes one or more surfaces for retaining one or more microorganisms upon the flow of said at least the portion of said fluid through said third module.

22. The method of claim 21, wherein said first fluid flow path is separate from said second fluid flow path.

23. The method of claim 21, wherein said one or more surfaces are part of one or more plates.

24. The method of claim 21, wherein said first module and second module are separable from one another.

25. The method of claim 21, wherein said second module and third module are separable from one another.

26. The method of claim 21, wherein said first module includes one or more internal impellers for facilitating fluid flow through said first and second modules.

27. The method of claim 26, wherein said one or more internal impellers are coupled to an external impeller that is external to said first, second and third modules, wherein said external impeller imparts rotational motion to said one or more internal impellers upon fluid flow through or adjacent to said external impeller.

28. The method of claim 21, wherein directing said fluid stream from said first module to said second module further comprises directing said fluid stream through a fourth module disposed between said first and second modules.

29. The method of claim 28, wherein said fourth module includes an optical window for permitting electromagnetic radiation for coming in contact with at least a portion of said fluid stream.
